(19) United States
(12) Patent Application Publication
(10) Pub. No.: US 2017/0233774 A1
(43) Pub. Date: Aug. 17, 2017

(54) MICROORGANISM CO-CULTURE SYSTEM AND USES OF THE SAME

(71) Applicant: Green Cellulosity Corporation, Hsinchu City (TW)

(72) Inventors: Chang-Chieh CHEN, Hsinchu City (TW); Cheng-Hao LIU, Hsinchu City (TW); Shih-Chan TSENG, Hsinchu City (TW); Ying-Ching SU, Hsinchu City (TW)

(73) Assignee: Green Cellulosity Corporation, Hsinchu City (TW)

(21) Appl. No.: 15/445,127

(22) Filed: Feb. 28, 2017

Related U.S. Application Data
(62) Division of application No. 14/845,588, filed on Sep. 4, 2015, now abandoned.
(60) Provisional application No. 62/046,335, filed on Sep. 5, 2014.

(51) Int. Cl. C12P 7/52; C12P 7/16

(52) U.S. Cl. CPC C12P 7/52; C12P 7/16; Y02E 50/10

(57) ABSTRACT

A method of producing butyric acid, comprising:
(a) providing a microorganism co-culture system comprising an air-tight container and the following (1) to (3) contained in the air-tight container:
   (1) a substrate, comprising a saccharide;
   (2) at least one of a first strain and a second strain, wherein the first strain is able to fix a carbon oxide and the second strain is able to fermentatively metabolize an amino acid, and wherein the first strain produces a first metabolite in the fermentation, the second strain produces a second metabolite in the fermentation, and each of the first metabolite and the second metabolite comprises acetic acid; and
   (3) a third strain, being able to metabolize the saccharide, the first metabolite and the second metabolite in the fermentation to produce butyric acid and a metabolic byproduct in fermentation, wherein the metabolic byproduct comprises carbon oxide and hydrogen,
   wherein, when the second strain is present in the co-culture system, the substrate further comprises an amino acid; and
(b) keeping the microorganism co-culture system under an anaerobic atmosphere to perform the fermentation and providing a fermentation product.

### BACKGROUND

This is a division of application Ser. No. 14/845,588, filed Sep. 4, 2015, and claims the benefit of U.S. provisional application No. 62/046,335, filed Sep. 5, 2014, all of which are incorporated herein by reference.

Technical Field

The present invention relates to a microorganism co-culture system and its uses, especially to the use of the co-culture system in the production of an organic compound (e.g., butyric acid and butanol). Specifically, microorganisms in the co-culture system of the present invention can interactively use the metabolites and metabolic byproducts produced in the fermentation, so as to increase the production efficiency and the carbon conversion rate of the entire fermentation.

Descriptions of Related Art

As of the early 20th century and along with the development of biofuel, microorganisms such as bacteria, yeasts and fungi have been being widely used in fermentation industry to convert biomass material into a more valuable organic compound such as an organic acid or an alcohol. Among the microorganism fermentation processes for the production of an organic compound, the acetone-butanol-ethanol (ABE) fermentation process (as shown in FIG. 1) is the most wildly used one. In the ABE fermentation pathway, with the use of microorganism, saccharide-containing material (e.g., corns, potatoes, syrups, etc.) can be converted into pyruvate that is to be further converted into acetyl-CoA to produce a more valuable organic compound such as acetic acid, ethanol, butyric acid, or butanol.

However, a carbon oxide (e.g., carbon dioxide) would be released during the conversion of pyruvate into acetyl-CoA (as shown in FIG. 1) and this causes unnecessary carbon loss. According to known processes, the highest carbon conversion rate of the ABE fermentation pathway is only about 66%, which leads to a poor yield of organic compound and makes unnecessary waste of cost and resource.

In view of the aforementioned problems of cost and resource waste, persons in the field have been endeavoring to breed and improve the strains of fermentation microorganisms. With respect to single microorganism fermentation processes, WO 2009/154624 A1 disclosed a fermentation process using engineered Clostridium tyrobutyricum, wherein an enhanced specificity of product was achieved by knocking-out the genes related to the synthesis of acetic acid in the ABE fermentation pathway (pta, ack); and US 2008/0248540A1 disclosed a fermentation process for the production of butyric acid by using Clostridium tyrobutyricum, and the butyric acid was converted into butanol by chemical reaction. The aforementioned two processes, however, are of low economic benefit for failing to increase the yield effectively. As for multi-microorganism fermentation processes, U.S. Pat. No. 8,420,359 B2 disclosed a fermentation system combining the lactic acid fermentation and the ABE fermentation, wherein the metabolites produced in the lactic acid fermentation (i.e., lactic acid) was used as a co-substrate for the ABE fermentation so as to increase the amount of the main product (i.e., butanol); U.S. Pat. No. 8,293,509 B2 disclosed a method of producing butanol with the use of a double bioreactor system, wherein two different microorganism bioreactors were disposed in the system, and particular by-product was recycled and reused by connecting the two bioreactors. The aforementioned two fermentation systems, however, are not ideal due to the necessity of using two or more bioreactors that need to be controlled respectively. The present invention is directed to the above needs.

### SUMMARY

The inventors have completed a microorganism co-culture system, wherein the microorganisms included in the system can live in a syntrophic relationship stably, i.e., the microorganisms can interactively use the metabolites and metabolic byproducts produced in the fermentation and are in a complementary relationship (as shown in FIGS. 2A, 2B, 2C). With the use of the system in a fermentation, various feedstocks could be converted into an organic compound such as butyric acid and butanol, and the needs of using the feedstocks efficiently, reducing unnecessary carbon loss, and providing a good yield of the target product could be fulfilled.

Thereof, an objective of the present invention is to provide a microorganism co-culture system, comprising:
(1) a substrate, comprising a saccharide;
(2) at least one of a first strain and a second strain, wherein the first strain is able to fix a carbon oxide and the second strain is able to fermentatively metabolize an amino acid, and wherein the first strain produces a first metabolite in the fermentation and the second strain produces a second metabolite in the fermentation; and
(3) a third strain, being able to metabolize the saccharide, the first metabolite and the second metabolite in the fermentation to produce butyric acid and/or butanol,
   wherein, when the second strain is present in the co-culture system, the substrate further comprises an amino acid. Preferably, the microorganism co-culture system further comprises a co-substrate, and preferably, the co-substrate is at least one of lactic acid and gaseous substrate.

Another objective of the present invention is to provide a method of producing butyric acid, comprising: providing the above microorganism co-culture system, wherein the metabolite of the third strain in the fermentation comprises butyric acid; and keeping the microorganism co-culture system under an anaerobic atmosphere to perform the fermentation and providing a fermentation product. Preferably, the method further comprises conducting a separation and purification procedure on the fermentation product.

Yet another objective of the present invention is to provide a method of producing butanol, comprising: providing the above microorganism co-culture system; keeping the microorganism co-culture system under an anaerobic atmosphere to perform the fermentation and provide a fermentation product; and optionally conducting a chemical conversion reaction to convert butyric acid into butanol. Preferably, the method further comprises conducting a separation and purification procedure on the fermentation product before conducting the chemical conversion reaction.

The detailed technology and preferred embodiments implemented for the present invention are described in the following paragraphs accompanying the appended drawings for people skilled in this field to well appreciate the features of the claimed invention.

### BRIEF DESCRIPTION OF THE DRAWINGS

FIG. 1 is a schematic diagram illustrating the acetone-butanol-ethanol (ABE) fermentation pathway, wherein \textcircled{1} is the EMP pathway; \textcircled{2} is pyruvate-ferredoxin oxidoreductase; \textcircled{3} is acetyl CoA-acetyl transferase/thiolase; \textcircled{4} is β-hydroxy butyryl CoA dehydrogenase; \textcircled{5} is crotonase; \textcircled{6} is butyryl CoA dehydrogenase; \textcircled{7} is phosphotransbutyrylase; \textcircled{8} is butyrate kinase; \textcircled{9} is butyraldehyde dehydrogenase; \textcircled{10} is butanol dehydrogenase; \textcircled{11} is phosphotransacetylase; \textcircled{12} is acetate kinase; \textcircled{13} is acetaldehyde dehydrogenase; \textcircled{14} is ethanol dehydrogenase; \textcircled{15} is CoA transferase; \textcircled{16} is acetoacetate decarboxylase; \textcircled{17} is ferredoxin-NAD(P)⁺ reductase; \textcircled{18} is hydrogenase; \textcircled{19} is butyryl CoA-acetate transferase; \textcircled{20} is lactate dehydrogenase;

FIG. 2A is a schematic diagram of an embodiment of the microorganism co-culture system according to the present invention, illustrating the interactive utilization of the metabolites and metabolic byproducts produced by the first strain and the third strain;

FIG. 2B is a schematic diagram of another embodiment of the microorganism co-culture system according to the present invention, illustrating the interactive utilization of the metabolites and metabolic byproducts produced by the second strain and the third strain;

FIG. 2C is a schematic diagram of another embodiment of the microorganism co-culture system according to the present invention, illustrating the interactive utilization of the metabolites and metabolic byproducts produced by the first strain, second strain and the third strain;

FIG. 3 is a schematic diagram illustrating the metabolic pathway that carbon oxides were recaptured by the first strain due to its ability of fixing carbon oxides and back to the fermentation to produce acetic acid (acetate);

FIG. 4A is a schematic diagram illustrating the metabolic pathway that carbohydrate serves as the carbon source of the third strain to produce butyric acid (butyrate); and

FIG. 4B is a schematic diagram illustrating the metabolic pathway that carbohydrate or organic acid serves as the carbon source of the third strain to produce butyric acid (butyrate).

### DESCRIPTION OF THE PREFERRED EMBODIMENT

The following will describe some embodiments of the present invention in detail. However, without departing from the spirit of the present invention, the present invention may be embodied in various embodiments and should not be limited to the embodiments described in the specification. In addition, unless otherwise indicated herein, the expressions “a,” “an”, “the”, or the like recited in the specification of the present invention (especially in the claims) are intended to include the singular and plural forms. Furthermore, the terms “about”, “approximate” or “almost” used in the specification substantially represented within ±20% of the stated value, preferably within ±10%, and more preferably within ±5%.

In the present invention, the term “microorganism” refers to an organism that is invisible to naked eyes (e.g., bacteria and fungi) and includes the wild type present in nature and mutant type induced by any factors (e.g., natural factor or artificial factor). The term “fermentation” refers to a process for metabolizing a substrate by a microorganism to produce an organic compound. The term “medium” refers to a composition providing nutrients and conditions (e.g., pH value, humidity, etc.) essential to the growth and replication of a microorganism. In general, the composition of the medium would be adjusted in accordance with the strain type of the microorganism to be incubated. For instance, adjustment onto the medium could be made by adding one or more of HCl, NaOH, NH₄OH, (NH₄)₂SO₄, NH₄Cl, CH₃COONH₄, K₂HPO₄, KH₂PO₄, NaH₂PO₃, Na₂HPO₃, citric acid, MgSO₄.7H₂O, FeSO₄.7H₂O, or MnSO₄.7H₂O so as to provide a medium with a desired pH value (e.g., pH 6) and/or desired physiochemical or physiological properties. The term “substrate” refers to a material that can be utilized during the fermentation of a microorganism, and thus, enters the metabolic pathway of the fermentation and then converts into other substance(s). The term “carbon oxide” refers to carbon monoxide, carbon dioxide, or a combination thereof.

Unless specifically indicated, the chemical names recited in the specification include all their isomer forms. Examples of the isomer forms include, but are not limited to, enantiomers, diastereomers and conformational isomers. For instance, the terms “lactic acid”, “glucose”, “xylose” and “galactose” all include their D-form and L-form isomers. Furthermore, when a saccharide can present in both open ring form and ring form at the same time, the chair form of its conformation isomer and its α, β isomers are all included.

In this specification, the term “carbon conversion rate” of a fermentation refers to the ratio between the total carbon number of the produced organic compound and the total carbon number of the consumed carbon source in the fermentation, and is calculated by Formula 1 as follows:

$$carbon conversion rate = \frac{\begin{matrix}the total carbon number of \\ produced organic compound\end{matrix}}{\begin{matrix}the total carbon number \\ of consumed carbon source\end{matrix}} \times 100 \% \qquad Formula 1$$

Known improvements of fermentation systems primarily focus on single bioreactor fermentation wherein one single strain is used, or on a system of multiple bioreactors wherein each strain conducts fermentation in an individual bioreactor and several bioreactors are connected to provide the system. Different from the prior art, the present invention provides a microorganism co-culture system, comprising:
(1) a substrate, comprising a saccharide;
(2) at least one of a first strain and a second strain, wherein the first strain is able to fix a carbon oxide and the second strain is able to fermentatively metabolize an amino acid, and wherein the first strain produces a first metabolite in the fermentation and the second strain produces a second metabolite in the fermentation; and
(3) a third strain, being able to metabolize the saccharide, the first metabolite and the second metabolite in the fermentation to produce butyric acid and/or butanol,
   wherein, when the second strain is present in the co-culture system, the substrate further comprises an amino acid. Preferably, the microorganism co-culture system further comprises a co-substrate, and preferably, the co-substrate is at least one of lactic acid and gaseous substrate.

In the microorganism co-culture system of the present invention, the substrate comprises a saccharide. Examples of suitable saccharide (also called “carbohydrate”) include, but are not limited to, monosaccharides (e.g., glucose, fructose, galactose, mannose, arabinose, lyxose, ribose, xylose, ribulose, xylulose, allose, altrose, gulose, idose, talose, psicose, sorbose, tagatose); disaccharides (e.g., sucrose, maltose, lactose, lactulose, trehalose, cellobiose); oligosaccharides (e.g., stachyose, maltotriose, maltotetrose, maltopentaose); and polysaccharides (e.g., starch, cellulose, glycogen, cyclodextrin, arabinoxylans, guar gum, gum arabic, chitin, gum, alginate, pectin, gellan). In some embodiments of the present invention, a substrate containing glucose or xylose was used as the carbon source for the fermentation.

In the process of growth and replication of a microorganism, amino acid is typically served as a nitrogen source for protein synthesis. However, different from such use, in the microorganism co-culture system according to the present invention, the amino acid, if any, contained in the substrate is used as a carbon source for the fermentation, and is metabolized to other organic compound(s). Examples of suitable sources of the amino acid include, but are not limited to, yeast extract, protein hydrolysate, peptone, corn steep liquor, whey, soybean meal, fish meal, meat bone meal, yeast powder, and soybean powder. In some embodiments of the present invention, a peptone-containing substrate was used to provide the carbon source for the fermentation.

In the microorganism co-culture system in accordance with the present invention, the presence of at least one of the first strain being able to fix a carbon monoxide and the second strain being able to metabolize an amino acid in the fermentation is required. In other words, in the microorganism co-culture system in accordance with the present invention, it is acceptable that the first strain is present and the second strain is absent, the second strain is present and the first strain is absent, or both the first strain and the second strain are present. When the second strain is present in the microorganism co-culture system in accordance with the present invention, the employed substrate further comprises an amino acid serving as the carbon source for the second strain in the fermentation.

The first strain can be any microorganism that is capable of fixing a carbon oxide. As used herein, “fixing carbon oxide” refers to the process of converting a carbon oxide into an organic compound by biochemical reaction. For instance, it is known that there are many microorganisms in the nature that can fix a carbon oxide present in living environment and convert the carbon oxide into acetyl-CoA through the Wood-Ljungdahl (WL) pathway (as shown in FIG. 3).

Examples of the strain being able to fix a carbon oxide through the Wood-Ljungdahl (WL) pathway include, but are not limited to, Clostridium coskatii, Clostridium ljungdahlii, Clostridium autoethanogenum, Clostridium ragsdalei, Terrisporobacter glycolicus, Clostridium carboxidivorans, Clostridium difficile, Clostridium aceticum, Moorella thermoacetica (previously known as Clostridium thermoaceticum), Methanobacterium thermoautotrophicum, Desulfobacterium autotrophicum, Clostridium sticklandii, Clostridium thermoautotrophicum, Clostridium formicoaceticum, Clostridium magnum, Acetobacterium carbinolicum, Acetobacterium kivui, Acetobacterium woodii, Acetitomaculum ruminis, Acetoanaerobium noterae, and Acetobacterium bakii. In addition to the above wild-type strains, the strain being able to fix a carbon oxide through the Wood-Ljungdahl (WL) pathway can be an engineered strain obtained by a genetic engineering procedure, as long as the metabolic pathway of the strain includes the WL pathway and the strain is able to fix a carbon oxide. For instance, for a strain whose metabolic pathway does not include the WL pathway or includes only part of the WL pathway, a gene of the WL pathway could be inserted into the strain by genetic engineering to render the strain to be able to fix a carbon oxide.

The above strains being able to fix a carbon oxide through the WL pathway can be used as the first strain in the microorganism co-culture system in accordance with the present invention. Preferably, the first strain is at least one of Clostridium coskatii, Clostridium ljungdahlii, Clostridium autoethanogenum, Clostridium ragsdalei, Terrisporobacter glycolicus, and Clostridium scatologenes.

When the microorganism co-culture system in accordance with the present invention is used in a fermentation, the first strain is able to fix a carbon oxide and produce a first metabolite that comprises acetic acid. For example, in some embodiments of the present invention, Clostridium ljungdahlii, Terrisporobacter glycolicus, or Clostridium scatologenes was used as the first strain to fix a carbon oxide and produce acetic acid in the fermentation.

In the microorganism co-culture system in accordance with the present invention, the second strain can be any microorganism capable of metabolizing an amino acid in the fermentation. As used herein, “metabolizing an amino acid in the fermentation” refers to that an amino acid is used as a substrate of the fermentation and is metabolized and converted into other organic compound(s). In the microorganism co-culture system in accordance with the present invention, the use of amino acid is different from its known use. The conventional use of amino acid is to serve as the nitrogen source needed in protein synthesis. However, “metabolizing an amino acid in the fermentation” herein refers to that the amino acid is served as the carbon source for the second strain in the fermentation, and is metabolized and used. Examples of microorganisms suitable to be used as the second strain for the microorganism co-culture system in accordance with the present invention can be the amino acid metabolizing strains described in the following articles: The amino acid-fermenting clostridia. J Gen Microbiol. 67(1):47-56 (1971); Enumeration of amino acid fermenting bacteria in the human large intestine: effects of pH and starch on peptide metabolism and dissimilation of amino acids. FEMS Microbiol Ecol. 15(4): 355-368 (1998); and The first 1000 cultured species of the human gastrointestinal microbiota. FEMS Microbiol Rev. 38(5):996-1047 (2014), which are entirely incorporated herein by reference. Preferred examples of the second strain include, but are not limited to, Clostridium cadaveris, Clostridium sporogenes, Clostridium sticklandii, Clostridium propionicum, Clostridium botulinum, and Clostridium pasteurianum.

When the microorganism co-culture system in accordance with the present invention is used in fermentation, the second strain can metabolize an amino acid and produce a second metabolite that comprises acetic acid. In addition to acetic acid, byproducts such as carbon oxides and hydrogen could be produces by the second strain. For example, in some embodiments of the present invention, Clostridium cadaveris or Clostridium sporogenes was used as the second strain in the microorganism co-culture system to metabolize an amino acid and produce acetic acid and minor butyric acid, together with carbon oxides and hydrogen as the byproducts in the fermentation. Specifically, in some embodiments of the present invention, the Clostridium cadaveris ITRI04005 disclosed in U.S. patent application Ser. No. 14/794,341 could be used as the second strain in the microorganism co-culture system in accordance with the present invention, the said strain is deposited at German Collection of Microorganisms and Cell Cultures (Deutsche Sammlung von Mikroorganismen and Zellkulturen GmbH, DSMZ) under the accession number DSM 32078, and deposited at Food Industry Research and Development Institute in Taiwan under the accession number BCRC 910680.

In another aspect, in the microorganism co-culture system in accordance with the present invention, any microorganism being able to metabolize at least one of the following substances to produce butyric acid and/or butanol can serve as the third strain for the co-culture system: (i) saccharide, (ii) the first metabolite produced by the first strain in the fermentation, and (iii) the second metabolite produced by the second strain in the fermentation.

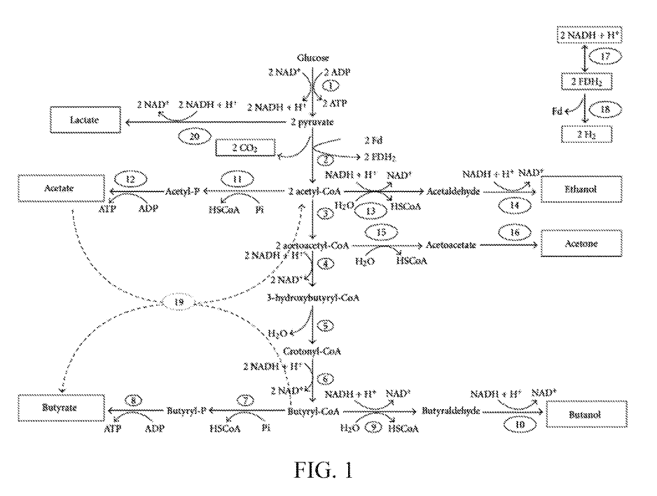

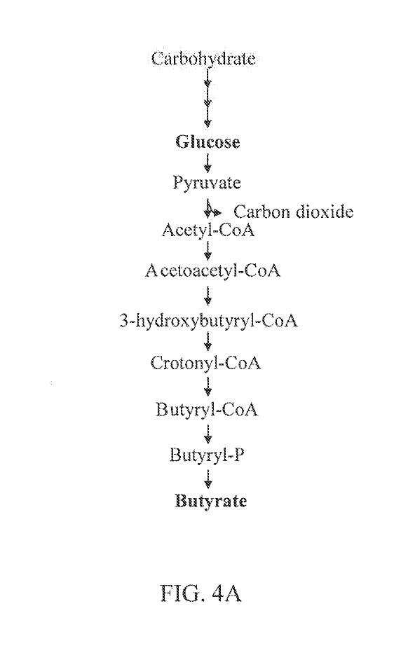

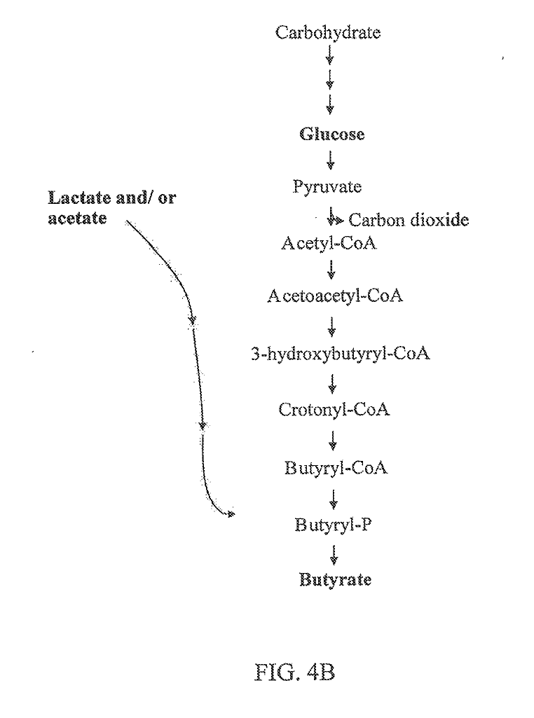

For example, the third strain can be a strain being able to conduct fermentation through the acetone-butanol-ethanol (ABE) pathway (as shown in FIGS. 1, 4A, and 4B), and could be such as Clostridium sp., but is not limited thereto. Other microorganisms suitable as the third strain and able to produce butyric acid and/or butanol in the fermentation include, but are not limited to, Anaerostipes butyraticus, Anaerostipes caccae, Anaerostipes sp., Butyrivibrio crossotus, Butyrivibrio fibrisolvens, Butyrivibrio hungatei, Butyrivibrio proteoclasticus, Clostridiales sp., Coprococcus ART55/1, Coprococcus catus, Coprococcus comes, Coprococcus eutactus, Eubacterium biforme, Eubacterium cellulosolvens, Eubacterium dolichum, Eubacterium hadrum, Eubacterium hallii, Eubacterium L2-7, Eubacterium limosum, Eubacterium oxidoreducens, Eubacterium ramulus, Eubacterium rectale, Eubacterium saburreum, Eubacterium A2-194, Eubacterium ventriosum, Lachnospiraceae bacterium, Lachnospiraceae sp., Moryella indoligenes, Parasporobacterium paucivorans, Pseudobutyrivibrio ruminis, Pseudobutyrivibrio xylanivorans, Roseburia cecicola, Roseburia faecis, Roseburia hominis, Roseburia intestinalis, Roseburia inulinivorans, Sporobacterium olearium, Anerococcus Octavius, Peptoniphilus asaccharolyticus, Peptoniphilus, duerdenii, Peptoniphilus harei, Peptoniphilus lacrimalis, Peptoniphilus indolicus, Peptoniphilus ivorii, Peptoniphilus sp., Sedimentibacter hydroxybenzoicus, Anaerovorax odorimutans, Filifactor alocis, Eubacterium barkeri, Eubacterium infirmum, Eubacterium minutum, Eubacterium nodatum, Eubacterium sulci, Eubacterium moniliforme, llyobacter delafieldii, Oxobacter pfenningii, Sarcina maxima, Thermobrachium celere, Butyricicoccus pullicaecorum, Eubacterium A2-207, Gemmiger formicilis, Anaerobaculum mobile, Pelospora glutarica, Thermoanaerobacter yonseiensis, Eubacterium cylindroides, Eubacterium saphenum, Eubacterium tortuosum, Eubacterium yurii margaretiae, Peptococcus anaerobius, Peptococcus niger, Sporotomaculum hydroxybenzoicum, Acidaminococcus intestine, Acidaminococcus fermentans, Acidaminococcus sp., Megasphaera elsdenii, Megasphaera genomosp, Megasphaera micronuciformis, Halanaerobium saccharolyticum, Brachyspira intermedia, Brachyspira alvinipulli, Shuttleworthia satelles Anaerococcus hydrogenalis, Anaerococcus lactolyticus, Anaerococcus prevotii, Anaerococcus tetradius, Anaerococcus vaginalis, Alkaliphilus metalliredigens, Alkaliphilus oremlandii, Anaerofustis stercorihominis, Pseudoramibacter alactolyticus, Anaerotruncus colihominis, Faecalibacterium cf. prausnitzii, Faecalibacterium prausnitzii, Ruminococcaceae bacterium, Subdoligranulum variabile, Thermoanaerobacterium thermosaccharolyticum, Carboxydibrachium pacificum, Carboxydothermus hydrogenoformans, Thermoanaerobacter tengcongensis, Thermoanaerobacter wiegelii, Erysipelotrichaceae bacterium, Carnobacterium sp., Desmospora sp., Acetonema longum, Thermosinus carboxydivorans, Natranaerobius thermophiles, Halanaerobium praevalens, Symbiobacterium thermophilum, Stackebrandtia nassauensis, Intrasporangium calvum, Janibacter sp., Micromonospora aurantiaca, Micromonospora sp. Salinispora arenicola, Salinispora tropica, Verrucosispora maris, Kribbella flavida, Nocardioidaceae bacterium, Nocardioides sp., Thermomonospora curvata, Haloplasma contractile, Desulfurispirillum indicum, Deferribacter desulfuricans, Rhodoferax ferrireducens, and Stigmatella aurantiaca. In addition to the above wild-type strains, the microorganism being able to produce butyric acid and/or butanol in fermentation can also be an engineered strain, as long as the strain is able to produce butyric acid and/or butanol in the fermentation. For instance, for a strain whose metabolic pathway does not include the ABE pathway or includes only part of the ABE pathway, a gene related to the ABE pathway could be inserted into the strain by genetic engineering to render the strain to be able to produce butyric acid and/or butanol by fermentation.

Preferably, the third strain is a strain of Clostridium sp. More preferably, the third strain is at least one of Clostridium tyrobutyricum, Clostridium butyricum, Clostridium beijerinckii, Clostridium acetobutylicum, Clostridium argentinense, Clostridium aurantibutyricum, Clostridium botulinum, Clostridium carboxidivorans, Clostridium cellulovorans, Clostridium cf. saccharolyticum, Clostridium difficile, Clostridium kluyveri, Clostridium novyi, Clostridium paraputrificum, Clostridium pascui, Clostridium peptidivorans, Clostridium perfringens, Clostridium scatologenes, Clostridium schirmacherense, Clostridium sticklandii, Clostridium subterminale SB4, Clostridium symbiosum, Clostridium tetani, Clostridium tepidiprofundi, Clostridium tertium, Clostridium tetanomorphum, and Clostridium thermopalmarium.

When the microorganism co-culture system in accordance with the present invention is used in a fermentation, the third strain is able to metabolize at least one of the following substances to produce butyric acid and/or butanol: (i) saccharide, (ii) the first metabolite produced by the first strain in the fermentation, and (iii) the second metabolite produced by the second strain in the fermentation. The fermentation of the third strain will additionally produce byproducts such as carbon oxides and hydrogen. For example, in some embodiments of the microorganism co-culture system in accordance with the present invention, Clostridium tyrobutyricum or Clostridium beijerinckii was served as the third strain to perform the above fermentation to produce butyric acid (when Clostridium tyrobutyricum was used) or butyric acid and butanol (when Clostridium beijerinckii was used), together with carbon oxides and hydrogen as byproducts.

In a conventional mixed-strain fermentation system, externally introduced syngas is essential for running the system (see such as WO 2014/113209 A1, which is entirely incorporated herein by reference). However, in the microorganism co-culture system in accordance with the present invention, an externally introduced gaseous substrate (e.g., syngas) is not essential because the carbon oxides produced by the second strain and/or the third strain in the fermentation can be captured by the first strain through its carbon oxide fixation ability and be used in the steps of fermentation, so as to efficiently use carbon source and reduce unnecessary carbon source loss due to such a complementary relationship among different strains.

Optionally, acetic acid could be externally added into the microorganism co-culture system in accordance with the present invention to provide the carbon source for the third strain in the fermentation (such as shown in FIG. 4B). Alternatively, the microorganism co-culture system can further comprise a co-substrate to provide additional carbon source to further increase the amount of the target organic compound (such as butyric acid and butanol). The co-substrate can be any suitable carbon compound, as long as it has no adverse effect on the strains, the performance of carbon oxide fixation, or the fermentation. Preferred examples of the carbon compound co-substrate include, but are not limited to, lactic acid, gaseous substrate, or a combination thereof, wherein the gaseous substrate can be at least one of syngas and industrial waste gas.

In the microorganism co-culture system in accordance with the present invention, when a saccharide-containing substrate is used and lactic acid is used as the co-substrate, a substrate mixture is provided by using 1 to 10 parts by weight of co-substrate per part by weight of saccharide. In an embodiment of the present invention, a substrate mixture is provided by mixing a glucose-containing substrate and lactic acid, wherein the weight ratio of glucose: lactic acid was about 1:1 to 1:10.

In the microorganism co-culture system in accordance with the present invention, the microorganism strains served as the first strain, the second strain, and the third strain are different from one another. Specifically, when Clostridium sticklandii is used as the second strain in the co-culture system, the first and the third strain are not Clostridium sticklandii; when Clostridium botulinum is used as the second strain in the co-culture system, the first and the third strain are not Clostridium botulinum; when Clostridium carboxidivorans is used as the first strain in the co-culture system, the second and the third strain are not Clostridium carboxidivorans; and when Clostridium difficile is used as the first strain in the co-culture system, the second and the third strain are not Clostridium difficile.

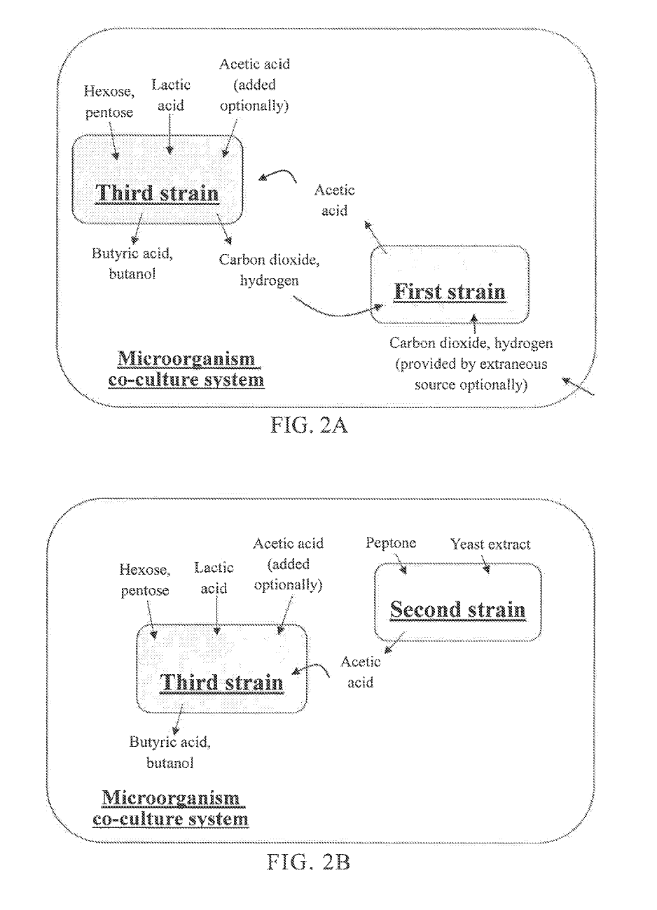

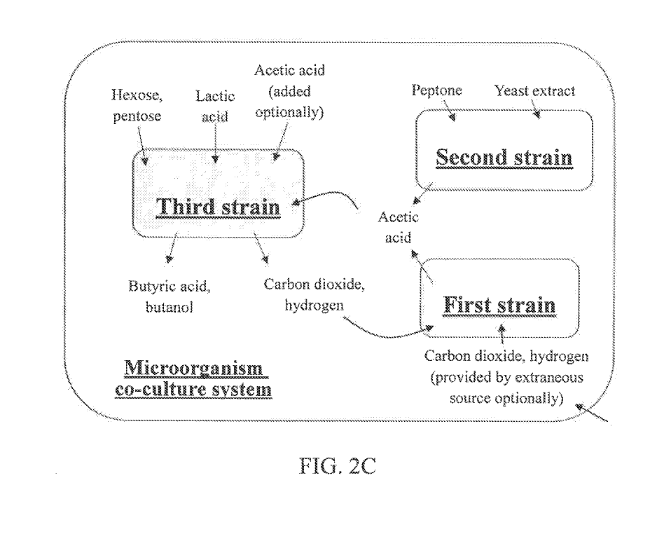

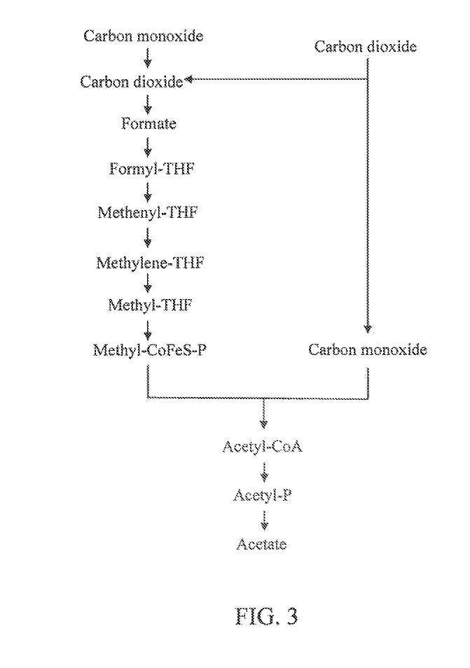

In the microorganism co-culture system in accordance with the present invention, since the carbon oxides (such as carbon dioxide) produced by the second strain and/or the third strain in the fermentation can be captured through the carbon oxide fixation by the first strain and back to the process of fermentation, the carbon resource can be used more efficiently, and the unnecessary carbon source loss can be reduced. Furthermore, since the second strain is able to fermentatively metabolize amino acid and the metabolite thus produced can be used by the third strain, such cycle is equivalent to an increase of additional carbon source. Moreover, in the fermentation, in addition to the saccharide contained in the substrate, the third strain can metabolize the first metabolite (such as acetic acid) produced by the first strain and the second metabolite (such as acetic acid) produced by the second strain; therefore, a good yield of target product (such as butyric acid and butanol) can be achieved (as shown in FIGS. 2A, 2B, 2C).

Accordingly, the present invention also provides a method of producing butyric acid, comprising: providing the above microorganism co-culture system, wherein the metabolite of the third strain in the fermentation comprises butyric acid; and keeping the microorganism co-culture system under an anaerobic atmosphere to perform the fermentation and providing a fermentation product. Preferably, the method of producing butyric acid in accordance with the present invention further comprises conducting a separation and purification procedure on the fermentation product to increase the purity of the butyric acid product. For example, the separation and purification procedure can be at least one of extraction, distillation, evaporation, ion-exchange, electrodialysis, filtration, and reverse osmosis, but is not limited thereto.

As shown in the following examples, with the use of the method of producing butyric acid in accordance with the present invention, a carbon conversion rate higher than the traditional theoretical value (i.e., 66%) could be achieved.

The present invention further provides a method of producing butanol, comprising: providing the above microorganism co-culture system; keeping the microorganism co-culture system under an anaerobic atmosphere to perform the fermentation and provide a fermentation product; and optionally conducting a chemical conversion reaction to convert butyric acid into butanol. For example, the chemical conversion reaction can be at least one of catalytic hydrogenation and esterification-hydrogenolysis, but is not limited thereto. Preferably, the method of producing butanol in accordance with the present invention further comprises conducting a separation and purification procedure on the fermentation product before conducting the chemical conversion reaction. For example, the separation and purification procedure can be at least one of extraction, distillation, evaporation, ion-exchange, electrodialysis, filtration, and reverse osmosis, but is not limited thereto.

In the method of producing butyric acid or butanol according to the present invention, the term “anaerobic atmosphere” refers to an atmosphere that contains less than 5 ppm (part per million) of oxygen, preferably less than 0.5 ppm of oxygen, and more preferably less than 0.1 ppm of oxygen. Any suitable method can be used to provide the desired anaerobic atmosphere. For example, but is not limited to, before the fermentation is performed, an inert gas (e.g., nitrogen, carbon dioxide) is introduced into the fermentation reactor to purge the reactor, and thus, provide the desired anaerobic atmosphere; alternatively, the fermentation is performed in an anaerobic operation box, wherein a palladium catalyst is used to catalyze the reaction of the oxygen in the box and the hydrogen in the anaerobic gas mixture to produce water, and thus, provide the desired anaerobic atmosphere.

In the method of producing butyric acid or butanol in accordance with the present invention, there is no particular limitation to the order of mixing the substrate and the strains. The substrate can be added at one time or in several batches before or during the fermentation, and the strains can be supplemented optionally. For instance, the substrate can be mixed with the strains at one time before performing the fermentation; the substrate also can be divided into two or more equal or unequal batches, and then the batches are separately added into the reactor before or during the fermentation.

Optionally, before the method of producing butyric acid or butanol starts, the strains used in the microorganism co-culture system can be pre-cultured until they grow into the log phase (i.e., when OD₆₀₀ is about 1.0 to 1.2). And such pre-cultured strains are used to perform fermentation to produce the desired butyric acid or butanol.

The present invention will be further illustrated in detail with specific examples as follows. However, the following examples are provided only for illustrating the present invention, and the scope of the present invention is not limited thereby.

### EXAMPLES

The materials used in the following examples comprise composition as follows:
(a) RCM (Reinforced Clostridial Medium) medium (purchased from Merck; comprising meat extract: 10 g/L; peptone: 10 g/L; yeast extract: 3 g/L; D (+) glucose: 5 g/L; NaCl: 5 g/L; sodium acetate: 3 g/L; L-cysteine hydrochloride: 0.5 g/L; starch: 1 g/L; agar: 0.5 g/L; pH 6.0).
(b) CGM (Clostridial Growth Medium) medium (yeast extract: 5 g/L; peptone: 5 g/L; (NH₄)₂SO₄: 3 g/L; K₂HPO₄: 1.5 g/L; MgSO₄.7H₂O: 0.6 g/L; FeSO₄.7H₂O: 0.03 g/L; Resazurin stock solution: 0.1% (weight/volume); pH 6.0).
(c) CSL-CGM (Corn steep liquor based CGM medium) medium ((NH₄)₂SO₄: 3 g/L; K₂HPO₄: 1.5 g/L; MgSO₄.7H₂O: 0.6 g/L; FeSO₄.7H₂O: 0.03 g/L; Resazurin stock solution: 0.1% weight/volume; CSL: 3.5, 5, 7, 10, 12, 15, or 18% (volume/volume); pH 6.0).
(d) mPETC medium (formulated in accordance with TW 201441366).
(e) P2 medium (yeast extract: 5 g/L; C₂H₃O₂NH₄: 2.2 g/L; MnSO₄.7H₂O: 0.01 g/L; NaCl: 1 g/L; MgSO₄.7H₂O: 0.2 g/L; FeSO₄.7H₂O: 0.01 g/L; p-amino benzoic acid (PABA): 1 mg/L; biotin: 0.01 mg/L; IVIES buffer: 39 g/L; pH 6.0).

In the following examples, an anaerobic atmosphere was provided in an air-tight container (e.g., air-tight serum bottle, centrifuge tube) by the following operations. The air-tight container and the rubber bung were covered with aluminum foil, and then sterilized under high temperature and high pressure (121° C., 1.2 atm) to exclude the interference of other microorganisms. After the sterilization was completed, the air-tight container was put in an oven to remove the residual moisture to prevent any microorganism contamination caused by the residual moisture. Thereafter, the dried air-tight container was transferred to an anaerobic operation box through the transfer box appended to the anaerobic operation box. After the sealing aluminum foil was slightly loosened, the palladium catalyst (purchased from Thermo Scientific, Inc., product number: BR0042) appended to the anaerobic operation apparatus was used to catalyze the reaction of the oxygen in the air-tight container and the hydrogen in the anaerobic gas mixture to produce water and to deplete the oxygen in the air-tight container, and thus, provide an anaerobic atmosphere.

In the following examples, all the mediums were treated as follows to be deoxygenated. First of all, the medium was prepared with desired composition. The prepared medium was sterilized under high temperature and high pressure (121° C., 1.5 atm) for 15 minutes, and then transferred into an anaerobic operation box through the transfer box appended to the anaerobic operation box before the medium cooled down to room temperature. Thereafter, the cap of the air-tight container in which the medium was kept was slightly loosened to release the steam contained therein. Then, with the use of the palladium catalyst appended to the anaerobic operation apparatus, the reaction of the oxygen in the air-tight container and the hydrogen in the anaerobic gas mixture was catalyzed to produce water such that deoxygenation of medium was performed. After the medium cooled down to room temperature, L-cysteine hydrochloride (0.5 g/L) was added therein to reduce the redox potential of the medium to a range suitable for microorganism such that a deoxygenated medium was provided.

### Example 1

### Use of a Microorganism Co-Culture System Containing a First Strain and a Third Strain in the Production of an Organic Acid

### Experiment 1-1: Strains

In Example 1, one of Clostridium ljungdahlii BCRC 17797 and Terrisporobacter glycolicus BCRC 14553, both are able to fix carbon oxide, was used as the first strain, and Clostridium tyrobutyricum BCRC 14535, which is able to metabolize saccharide or organic compound to produce organic acid (such as acetic acid and butyric acid) in fermentation, was used as the third strain.

### Experiment 1-2: Pre-Culture

(a) Clostridium ljungdahlii BCRC 17797: a single colony of this strain was selected, inoculated in 10 ml deoxygenated RCM medium being externally added with 10 g/L fructose, and incubated in an anaerobic incubator at 37° C. for 48 hours so as to let the OD₆₀₀ (the absorbance at a wavelength of 600 nm) of the strain reach about 1.0 to 1.2.
(b) Terrisporobacter glycolicus BCRC 14553/Clostridium tyrobutyricum BCRC 14535: a single colony of the strain was selected, inoculated in 10 ml deoxygenated RCM medium, and incubated in an anaerobic incubator at 37° C. for 14 hours to 16 hours so as to let the OD₆₀₀ (the absorbance at a wavelength of 600 nm) of the strain reach about 1.0 to 1.2.

### Experiment 1-3: Fermentation Tests

### Test 1-3-1

CGM medium was mixed with glucose to provide a medium mixture with a glucose concentration of 10 g/L (pH=6.0), and then the medium mixture was deoxygenated. Each of two air-tight serum bottles was injected with 60 ml of the above deoxygenated medium mixture.

Each of the pre-cultured Clostridium ljungdahlii BCRC 17797 and Clostridium tyrobutyricum BCRC 14535 prepared in Experiment 1.2 was inoculated into one of the above two air-tight serum bottles at about 30% inoculation rate; and the pre-cultured Clostridium tyrobutyricum BCRC 14535 prepared in Experiment 1.2 was inoculated into the other air-tight serum bottle at about 30% inoculation rate. The two air-tight serum bottles were then kept in an anaerobic incubator at 37° C. and samples were taken therefrom at 7 hour and 24 hours, respectively. The samples were analyzed by Agilent 1100 HPLC analysis in combination with Aminex HPX-87H (300×7.8 mm) column so as to calculate the consumption of glucose and the amounts of acetic acid and butyric acid in the above culture medium. In addition, the carbon conversion rates of butyric acid and organic acid were calculated. The results are shown in Table 1.

TABLE 1

| | | | | | Carbon | Carbon |
|---|---|---|---|---|---|---|
| | | | | | conversion | conversion |
| Incubation | | Consumption | Amount of | Amount of | rate of | rate of |
| time | | of glucose | acetic acid | butyric acid | butyric | organic |
| (hour) | strain | (g/L) | (g/L) | (g/L) | acid (%) | acid (%) |
| 7 | BCRC17797 + | 5.03 | 0.60 | 2.13 | 57.59 | 69.53 |
| | BCRC14535 | | | | | |
| | BCRC14535 | 4.40 | 0.22 | 1.69 | 52.31 | 57.38 |
| 24 | BCRC17797 + | 9.33 | 0.59 | 4.38 | 64.08 | 70.36 |
| | BCRC14535 | | | | | |
| | BCRC14535 | 9.33 | 0.40 | 3.85 | 56.24 | 60.51 |

As shown in Table 1, after the incubation of 7 hours, as compared with the system comprising Clostridium tyrobutyricum BCRC 14535 alone, the system comprising both Clostridium ljungdahlii BCRC 17797 and Clostridium tyrobutyricum BCRC 14535 was much better in the consumption rate of glucose (i.e., substrate). On the other hand, regardless the incubation time was 7 hours or 24 hours, the production rate of organic acid, carbon conversion rate of butyric acid, and carbon conversion rate of organic acid of the system comprising both Clostridium ljungdahlii BCRC 17797 and Clostridium tyrobutyricum BCRC 14535 were markedly higher than those of the system comprising Clostridium tyrobutyricum BCRC 14535 alone. The above results indicate that the Clostridium ljungdahlii BCRC 17797 and Clostridium tyrobutyricum BCRC 14535 co-culture system could provide a better utilization rate of substrate, a better yield of fermentation product, and a better carbon conversion rate.

### Test 1-3-2

CSL-CGM medium was mixed with glucose to provide a medium mixture with a glucose concentration of 12 g/L and a CSL concentration of about 3.5% (pH=6.0), and then the medium mixture was deoxygenated. Each of two air-tight serum bottles was injected with 60 ml of the above deoxygenated medium mixture.

Each of the pre-cultured Clostridium ljungdahlii BCRC 17797 and Clostridium tyrobutyricum BCRC 14535 prepared in the Experiment 1.2 was inoculated into one of the above two air-tight serum bottles at about 30% inoculation rate; and the pre-cultured Clostridium tyrobutyricum BCRC 14535 prepared in the Experiment 1.2 was inoculated into the other air-tight serum bottle at about 30% inoculation rate. The two air-tight serum bottles were then kept in an anaerobic incubator at 37° C. and samples were taken therefrom at 24 hours. The samples were analyzed by Agilent 1100 HPLC analysis in combination with Aminex HPX-87H (300×7.8 mm) column so as to calculate the consumptions of glucose and lactic acid and the amounts of acetic acid and butyric acid in the above culture medium. In addition, the carbon conversion rates of butyric acid and organic acid were calculated. The results are shown in Table 2.

TABLE 2

| | | | Amount | | Carbon | Carbon |
|---|---|---|---|---|---|---|
| | | Consumption | of | Amount | conversion | conversion |
| | Consumption | of | acetic | of | rate of | rate of |
| | of glucose | lactic acid | acid | butyric | butyric | organic |
| Strain | (g/L) | (g/L) | (g/L) | acid (g/L) | acid (%) | acid (%) |
| BCRC17797 + | 12.15 | 3.29 | 0.59 | 7.76 | 68.57 | 72.39 |
| BCRC14535 | | | | | | |
| BCRC14535 | 3.83 | 0.75 | 0 | 2.0 | 60.0 | 60.0 |

As shown in Table 2, as compared with the system comprising Clostridium tyrobutyricum BCRC 14535 alone, the system comprising both Clostridium ljungdahlii BCRC 17797 and Clostridium tyrobutyricum BCRC 14535 was much better in the consumption rates of glucose (i.e., substrate) and lactic acid (i.e., co-substrate), the production rate of organic acid, and the carbon conversion rates of butyric acid and organic acid. The above results indicate that the Clostridium ljungdahlii BCRC 17797 and Clostridium tyrobutyricum BCRC 14535 co-culture system could provide better utilization rates of substrate and co-substrate, a better yield of fermentation product, and a better carbon conversion rate. And, the carbon conversion rate of butyric acid was even higher than the maximum theoretical value of the conventional ABE fermentation (i.e., 66%).

### Test 1-3-3

CSL-CGM medium was mixed with glucose to provide a medium mixture with a glucose concentration of 10 g/L and a CSL concentration of about 5% (pH=6.0), and then the medium mixture was deoxygenated. Each of two air-tight serum bottles was injected with 60 ml of the above deoxygenated medium mixture.

Each of the pre-cultured Clostridium ljungdahlii BCRC 17797 and Clostridium tyrobutyricum BCRC 14535 prepared in the Experiment 1.2 was inoculated into one of the above two air-tight serum bottles at about 30% inoculation rate; and the pre-cultured Clostridium tyrobutyricum BCRC 14535 prepared in the Experiment 1.2 was inoculated into the other air-tight serum bottle at about 30% inoculation rate. The two air-tight serum bottles were then kept in an anaerobic incubator at 37° C. and samples were taken therefrom at 17 hours. The samples were analyzed by Agilent 1100 HPLC analysis in combination with Aminex HPX-87H (300×7.8 mm) column so as to calculate the consumptions of glucose and lactic acid and the amounts of acetic acid and butyric acid in the above culture medium. In addition, the carbon conversion rates of butyric acid and organic acid were calculated. The results are shown in Table 3.

TABLE 3

| | | | | | Carbon | Carbon |
|---|---|---|---|---|---|---|
| | | | | | conversion | conversion |
| | Consumption | Consumption | Amount of | Amount of | rate of | rate of |
| | of glucose | of lactic acid | acetic acid | butyric | butyric | organic |
| Strain | (g/L) | (g/L) | (g/L) | acid (g/L) | acid (%) | acid (%) |
| BCRC17797 + | 10.20 | 5.01 | 0.61 | 7.93 | 71.11 | 74.96 |
| BCRC14535 | | | | | | |
| BCRC14535 | 5.43 | 1.14 | 0.2 | 2.61 | 54.29 | 57.22 |

As shown in Table 3, as compared with the system comprising Clostridium tyrobutyricum BCRC 14535 alone, the system comprising both Clostridium ljungdahlii BCRC 17797 and Clostridium tyrobutyricum BCRC 14535 was much better in the consumption rates of glucose (i.e., substrate) and lactic acid (i.e., co-substrate), the production rate of organic acid, and the carbon conversion rates of butyric acid and organic acid. The above results indicate again that the Clostridium ljungdahlii BCRC 17797 and Clostridium tyrobutyricum BCRC 14535 co-culture system could provide better utilization rates of substrate and co-substrate, a better yield of fermentation product, and a better carbon conversion rate. And the carbon conversion rate of butyric acid was even higher than the maximum theoretical value of the conventional ABE fermentation (i.e., 66%).

### Test 1-3-4

CSL-CGM medium was mixed with glucose to provide a medium mixture with a glucose concentration of 9 g/L and a CSL concentration of about 7% (pH=6.0), and then the medium mixture was deoxygenated. Each of two air-tight serum bottles was injected with 60 ml of the above deoxygenated medium mixture.

Each of the pre-cultured Clostridium ljungdahlii BCRC 17797 and Clostridium tyrobutyricum BCRC 14535 prepared in the Experiment 1.2 was inoculated into one of the above two air-tight serum bottles at about 30% inoculation rate; and the pre-cultured Clostridium tyrobutyricum BCRC 14535 prepared in the Experiment 1.2 was inoculated into the other air-tight serum bottle at about 30% inoculation rate. The two air-tight serum bottles were then kept in an anaerobic incubator at 37° C. and samples were taken therefrom at 24 hours. The samples were analyzed by Agilent 1100 HPLC analysis in combination with Aminex HPX-87H (300×7.8 mm) column so as to calculate the consumptions of glucose and lactic acid and the amounts of acetic acid and butyric acid in the above culture medium. In addition, the carbon conversion rates of butyric acid and organic acid were calculated. The results are shown in Table 4.

TABLE 4

| | | | | | Carbon | Carbon |
|---|---|---|---|---|---|---|
| | | | | | conversion | conversion |
| | Consumption | Consumption | Amount of | Amount of | rate of | rate of |
| | of glucose | of lactic acid | acetic acid | butyric | butyric | organic |
| Strain | (g/L) | (g/L) | (g/L) | acid (g/L) | acid (%) | acid (%) |
| BCRC17797 + | 9.34 | 7.11 | 1.1 | 9.01 | 74.72 | 81.41 |
| BCRC14535 | | | | | | |
| BCRC14535 | 6.21 | 1.96 | 0 | 3.72 | 62.09 | 62.09 |

As shown in Table 4, as compared with the system comprising Clostridium tyrobutyricum BCRC 14535 alone, the system comprising both Clostridium ljungdahlii BCRC 17797 and Clostridium tyrobutyricum BCRC 14535 was much better in the consumption rates of glucose (i.e., substrate) and lactic acid (i.e., co-substrate), the production rate of organic acid, and the carbon conversion rates of butyric acid and organic acid. The above results also indicate that the Clostridium ljungdahlii BCRC 17797 and Clostridium tyrobutyricum BCRC 14535 co-culture system could provide better utilization rates of substrate and co-substrate, a better yield of fermentation product, and a better carbon conversion rate. And the carbon conversion rate of butyric acid was even higher than the maximum theoretical value of the conventional ABE fermentation (i.e., 66%).

### Test 1-3-5

A CSL-CGM medium with a CSL concentration of about 15% (pH=6.0) was prepared, and then the medium was deoxygenated. Thereafter, an air-tight serum bottle was injected with 60 ml of the above deoxygenated CSL-CGM medium.

Each of the pre-cultured Clostridium ljungdahlii BCRC 17797 and Clostridium tyrobutyricum BCRC 14535 prepared in Experiment 1.2 was inoculated into the above air-tight serum bottle at about 30% inoculation rate, respectively. The air-tight serum bottle was then kept in an anaerobic incubator at 37° C. and sample was taken therefrom at 24 hours. The sample was analyzed by Agilent 1100 HPLC analysis in combination with Aminex HPX-87H (300×7.8 mm) column so as to calculate the consumption of lactic acid and the amounts of acetic acid and butyric acid in the above culture medium. In addition, the carbon conversion rates of butyric acid and organic acid were calculated. The results are shown in Table 5.

TABLE 5

| | | | | Carbon | Carbon |
|---|---|---|---|---|---|
| | Consumption | Amount of | Amount of | conversion rate | conversion rate |
| | of lactic acid | acetic acid | butyric acid | of butyric acid | of organic acid |
| Strain | (g/L) | (g/L) | (g/L) | (%) | (%) |
| BCRC17797 + | 13.57 | 0 | 9.11 | 91.55 | 91.55 |
| BCRC14535 | | | | | |

As shown in Table 5, for the medium using the system comprising both Clostridium ljungdahlii BCRC 17797 and Clostridium tyrobutyricum BCRC 14535, even though only CSL (which contained protein, lactic acid, and minor saccharide) but not glucose (i.e., substrate) was added thereto, the production of butyric acid could also be detected and the carbon conversion rates of butyric acid and organic acid both reached 91.55%. The result indicates that the Clostridium ljungdahlii BCRC 17797 and Clostridium tyrobutyricum BCRC 14535 co-culture system could convert an amino acid or lactic acid into product such as butyric acid under a condition without glucose, and it could provide a good carbon conversion rate (much higher than the traditional theoretical value of 66%).

### Test 1-3-6

CGM medium was mixed with lactate to provide a medium mixture with a lactic acid concentration of 15 g/L (pH=6.0), and then the medium mixture was deoxygenated. Thereafter, an air-tight serum bottle was injected with 50 ml of the above deoxygenated medium mixture.

Each of the pre-cultured Terrisporobacter glycolicus BCRC 14553 and Clostridium tyrobutyricum BCRC 14535 provided in the Experiment 1.2 was inoculated into the above air-tight serum bottle at about 20% inoculation rate. The air-tight serum bottle was then kept in an anaerobic incubator at 37° C. and sample was taken therefrom at 120 hours. The sample was analyzed by Agilent 1100 HPLC analysis in combination with Aminex HPX-87H (300×7.8 mm) column so as to calculate the consumption of lactic acid and the amounts of acetic acid and butyric acid in the above culture medium. In addition, the carbon conversion rates of butyric acid and organic acid were calculated. The results are shown in Table 6.

TABLE 6

| | | | | Carbon | Carbon |
|---|---|---|---|---|---|
| | Consumption | Amount of | Amount of | conversion rate | conversion rate |
| | of lactic acid | acetic acid | butyric acid | of butyric acid | of organic acid |
| Strain | (g/L) | (g/L) | (g/L) | (%) | (%) |
| BCRC14553 + | 12.48 | 0.01 | 8.05 | 87.96 | 88.04 |
| BCRC14535 | | | | | |

As shown in Table 6, for the medium using the system comprising both Terrisporobacter glycolicus BCRC 14553 and Clostridium tyrobutyricum BCRC 14535, even though only lactic acid but not glucose (i.e., substrate) was added thereto, the production of butyric acid could also be detected and the carbon conversion rates of butyric acid and organic acid were 87.96% and 88.04%, respectively, and were both higher than the traditional theoretical value (i.e., 66%). The result indicates that the Terrisporobacter glycolicus BCRC 14553 and Clostridium tyrobutyricum BCRC 14535 co-culture system could convert lactic acid into product such as butyric acid under a condition without glucose, and it could provide a good carbon conversion rate (much higher than the traditional theoretical value of 66%).

### Example 2

### Use of a Microorganism Co-Culture System Containing a Second Strain and a Third Strain in the Production of an Organic Acid

### Experiment 2-1: Strains

In Example 2, one of Clostridium cadaveris BCRC 14511 and Clostridium sporogenes BCRC 11259, both are able to fermentatively metabolize amino acid, was used as the second strain, and Clostridium tyrobutyricum BCRC 14535, which is able to metabolize saccharide or organic compound to produce organic acid (such as acetic acid and butyric acid) in fermentation, was used as the third strain.

### Experiment 2-2: Pre-Culture

A single colony of each of the Clostridium cadaveris BCRC 14511, Clostridium sporogenes BCRC 11259, or Clostridium tyrobutyricum BCRC 14535 was selected, inoculated in 10 ml deoxygenated RCM medium, and incubated in an anaerobic incubator at 37° C. for 14 hours to 16 hours so as to let the OD₆₀₀ (the absorbance at a wavelength of 600 nm) of the strains reach about 1.0 to 1.2.

### Experiment 2-3: Fermentation Tests

### Test 2-3-1

CGM medium was mixed with glucose to provide a medium mixture with a glucose concentration of 10 g/L (pH=6.0), and then the medium mixture was deoxygenated. Each of two air-tight serum bottles was injected with 60 ml of the above deoxygenated medium mixture.

Each of the pre-cultured Clostridium cadaveris BCRC 14511 and Clostridium tyrobutyricum BCRC 14535 prepared in the Experiment 2.2 was inoculated into one of the above two air-tight serum bottles at about 30% inoculation rate; and the pre-cultured Clostridium tyrobutyricum BCRC 14535 prepared in the Experiment 2.2 was inoculated into the other air-tight serum bottle at about 30% inoculation rate. The two air-tight serum bottles were then kept in an anaerobic incubator at 37° C. and samples were taken therefrom at 7 hours or 24 hours. The samples were analyzed by Agilent 1100 HPLC analysis in combination with Aminex HPX-87H (300×7.8 mm) column so as to calculate the consumption of glucose and the amounts of acetic acid and butyric acid in the above culture medium. In addition, the carbon conversion rates of butyric acid and organic acid were calculated. The results are shown in Table 7.

TABLE 7

| | | | | | Carbon | Carbon |
|---|---|---|---|---|---|---|
| | | | | | conversion | conversion |
| Incubation | | Consumption | Amount of | Amount of | rate of | rate of |
| time | | of glucose | acetic acid | butyric | butyric | organic |
| (hour) | Strain | (g/L) | (g/L) | acid (g/L) | acid (%) | acid (%) |
| 7 | BCRC14511 + | 6.02 | 0.33 | 2.55 | 57.90 | 63.37 |
| | BCRC14535 | | | | | |
| | BCRC14535 | 4.40 | 0.22 | 1.69 | 52.31 | 57.38 |
| 24 | BCRC14511 + | 9.26 | 0.17 | 4.21 | 62.05 | 63.87 |
| | BCRC14535 | | | | | |
| | BCRC14535 | 9.33 | 0.40 | 3.85 | 56.24 | 60.51 |

As shown in Table 7, after the incubation of 7 hours, as compared with the system comprising Clostridium tyrobutyricum BCRC 14535 alone, the system comprising both Clostridium cadaveris BCRC 14511 and Clostridium tyrobutyricum BCRC 14535 was much better in the consumption rate of glucose (i.e., substrate) and the production rate of organic acid. On the other hand, regardless the incubation time was 7 hours or 24 hours, the carbon conversion rate of butyric acid and carbon conversion rate of organic acid of the system comprising both Clostridium cadaveris BCRC 14511 and Clostridium tyrobutyricum BCRC 14535 were markedly higher than those of the system comprising Clostridium tyrobutyricum BCRC 14535 alone. The above results indicate that the Clostridium cadaveris BCRC 14511 and Clostridium tyrobutyricum BCRC 14535 co-culture system could provide a better utilization rate of substrate, a better yield of fermentation product, and a better carbon conversion rate.

### Test 2-3-2

CGM medium was mixed with glucose and lactate to provide a medium mixture with a glucose concentration of 3 g/L and a lactic acid concentration of 7 g/L (pH=6.0), and then the medium mixture was deoxygenated. Each of two air-tight serum bottles was injected with 60 ml of the above deoxygenated medium mixture.

Each of the pre-cultured Clostridium cadaveris BCRC 14511 and Clostridium tyrobutyricum BCRC 14535 prepared in the Experiment 2.2 was inoculated into one of the above two air-tight serum bottles at about 30% inoculation rate; and the pre-cultured Clostridium tyrobutyricum BCRC 14535 prepared in the Experiment 2.2 was inoculated into the other air-tight serum bottle at about 30% inoculation rate. The two air-tight serum bottles were then kept in an anaerobic incubator at 37° C. and samples were taken therefrom at 24 hours. The samples were analyzed by Agilent 1100 HPLC analysis in combination with Aminex HPX-87H (300×7.8 mm) column so as to calculate the consumptions of glucose and lactic acid, the amounts of acetic acid and butyric acid in the above culture medium. In addition, the carbon conversion rates of butyric acid and organic acid were calculated. The results are shown in Table 8.

TABLE 8

| | | | | | Carbon | Carbon |
|---|---|---|---|---|---|---|
| | | | | | conversion | conversion |
| | Consumption | Consumption | Amount of | Amount of | rate of | rate of |
| | of glucose | of lactic acid | acetic acid | butyric | butyric | organic |
| Strain | (g/L) | (g/L) | (g/L) | acid (g/L) | acid (%) | acid (%) |
| BCRC14511 + | 3.1 | 6.4 | 0.2 | 5.2 | 74.64 | 76.75 |
| BCRC14535 | | | | | | |
| BCRC14535 | 3.1 | 3.7 | 0 | 3.0 | 60.16 | 60.16 |

As shown in Table 8, as compared with the system comprising Clostridium tyrobutyricum BCRC 14535 alone, the system comprising both Clostridium Clostridium cadaveris BCRC 14511 and Clostridium tyrobutyricum BCRC 14535 was much better in the consumption rate of lactic acid (i.e., co-substrate), the production rate of organic acid, and the carbon conversion rates of butyric acid and organic acid. The above results indicate that the Clostridium cadaveris BCRC 14511 and Clostridium tyrobutyricum BCRC 14535 co-culture system could provide a better utilization rate of co-substrate, a better yield of fermentation product, and a better carbon conversion rate. And the carbon conversion rate of butyric acid was even higher than the maximum theoretical value of the conventional ABE fermentation (i.e., 66%).

### Test 2-3-3

CSL-CGM medium was mixed with glucose to provide a medium mixture with a glucose concentration of 12 g/L and a CSL concentration of about 3.5% (pH=6.0), and then the medium mixture was deoxygenated. Each of two air-tight serum bottles was injected with 60 ml of the above deoxygenated medium mixture.

Each of the pre-cultured Clostridium cadaveris BCRC 14511 and Clostridium tyrobutyricum BCRC 14535 prepared in the Experiment 2.2 was inoculated into one of the above two air-tight serum bottles at about 30% inoculation rate; and the pre-cultured Clostridium tyrobutyricum BCRC 14535 prepared in the Experiment 2.2 was inoculated into the other air-tight serum bottle at about 30% inoculation rate. The two air-tight serum bottles were then kept in an anaerobic incubator at 37° C. and samples were taken therefrom at 24 hours. The samples were analyzed by Agilent 1100 HPLC analysis in combination with Aminex HPX-87H (300×7.8 mm) column so as to calculate the consumptions of glucose and lactic acid, the amounts of acetic acid and butyric acid in the above culture medium. In addition, the carbon conversion rates of butyric acid and organic acid were calculated. The results are shown in Table 9.

TABLE 9

| | | | | | Carbon | Carbon |
|---|---|---|---|---|---|---|
| | | | | | conversion | conversion |
| | Consumption | Consumption | Amount of | Amount of | rate of | rate of |
| | of glucose | of lactic acid | acetic acid | butyric | butyric | organic |
| Strain | (g/L) | (g/L) | (g/L) | acid (g/L) | acid (%) | acid (%) |
| BCRC14511 + | 9.7 | 2.65 | 0 | 5.8 | 64.04 | 64.04 |
| BCRC14535 | | | | | | |
| BCRC14535 | 3.83 | 0.75 | 0 | 2.0 | 60.0 | 60.0 |

As shown in Table 9, as compared with the system comprising Clostridium tyrobutyricum BCRC 14535 alone, the system comprising both Clostridium Clostridium cadaveris BCRC 14511 and Clostridium tyrobutyricum BCRC 14535 was much better in the consumption rates of glucose (i.e., substrate) and lactic acid (i.e., co-substrate), the production rate of organic acid, and the carbon conversion rates of butyric acid and organic acid. The above results indicate again that the Clostridium cadaveris BCRC 14511 and Clostridium tyrobutyricum BCRC 14535 co-culture system could provide better utilization rates of substrate and co-substrate, a better yield of fermentation product, and a better carbon conversion rate.

### Test 2-3-4

CSL-CGM medium was mixed with glucose to provide a medium mixture with a glucose concentration of 10 g/L and a CSL concentration of about 5% (pH=6.0), and then the medium mixture was deoxygenated. Each of two air-tight serum bottles was injected with 60 ml of the above deoxygenated medium mixture.

Each of the pre-cultured Clostridium cadaveris BCRC 14511 and Clostridium tyrobutyricum BCRC 14535 prepared in the Experiment 2.2 was inoculated into one of the above two air-tight serum bottles at about 30% inoculation rate; and the pre-cultured Clostridium tyrobutyricum BCRC 14535 prepared in the Experiment 2.2 was inoculated into the other air-tight serum bottle at about 30% inoculation rate. The two air-tight serum bottles were then kept in an anaerobic incubator at 37° C. and samples were taken therefrom at 17 hours. The samples were analyzed by Agilent 1100 HPLC analysis in combination with Aminex HPX-87H (300×7.8 mm) column so as to calculate the consumptions of glucose and lactic acid, the amounts of acetic acid and butyric acid in the above culture medium. In addition, the carbon conversion rates of butyric acid and organic acid were calculated. The results are shown in Table 10.

TABLE 10

| | | | | | Carbon | Carbon |
|---|---|---|---|---|---|---|
| | | | | | conversion | conversion |
| | Consumption | Consumption | Amount of | Amount of | rate of | rate of |
| | rate of | rate of lactic | acetic acid | butyric | butyric | organic |
| Strain | glucose (g/L) | acid (g/L) | (g/L) | acid (g/L) | acid (%) | acid (%) |
| BCRC14511 + | 9.86 | 3.09 | 0 | 6.31 | 66.44 | 66.44 |
| BCRC14535 | | | | | | |
| BCRC14535 | 5.43 | 1.14 | 0 | 2.61 | 54.17 | 54.17 |

As shown in Table 10, as compared with the system comprising Clostridium tyrobutyricum BCRC 14535 alone, the system comprising both Clostridium Clostridium cadaveris BCRC 14511 and Clostridium tyrobutyricum BCRC 14535 was much better in the consumption rates of glucose (i.e., substrate) and lactic acid (i.e., co-substrate), the production rate of organic acid, and the carbon conversion rates of butyric acid and organic acid. The above results also indicate that the Clostridium cadaveris BCRC 14511 and Clostridium tyrobutyricum BCRC 14535 co-culture system could provide better utilization rates of substrate and co-substrate, a better yield of fermentation product, and a better carbon conversion rate.

### Test 2-3-5

CSL-CGM medium was mixed with glucose to provide a medium mixture with a glucose concentration of 9 g/L and a CSL concentration of about 7% (pH=6.0), and then the medium mixture was deoxygenated. Each of two air-tight serum bottles was injected with 60 ml of the above deoxygenated medium mixture.

Each of the pre-cultured Clostridium cadaveris BCRC 14511 and Clostridium tyrobutyricum BCRC 14535 prepared in the Experiment 2.2 was inoculated into one of the above two air-tight serum bottles at about 30% inoculation rate; and the pre-cultured Clostridium tyrobutyricum BCRC 14535 prepared in the Experiment 2.2 was inoculated into the other air-tight serum bottle at about 30% inoculation rate. The two air-tight serum bottles were then kept in an anaerobic incubator at 37° C. and samples were taken therefrom at 24 hours. The samples were analyzed by Agilent 1100 HPLC analysis in combination with Aminex HPX-87H (300×7.8 mm) column so as to calculate the consumptions of glucose and lactic acid, the amounts of acetic acid and butyric acid in the above culture medium. In addition, the carbon conversion rates of butyric acid and organic acid were calculated. The results are shown in Table 11.

TABLE 11

| | | | | | Carbon | Carbon |
|---|---|---|---|---|---|---|
| | | | | | conversion | conversion |
| | Consumption | Consumption | Amount of | Amount of | rate of | rate of |
| | of glucose | of lactic acid | acetic acid | butyric | butyric | organic |
| Strain | (g/L) | (g/L) | (g/L) | acid (g/L) | acid (%) | acid (%) |
| BCRC14511 + | 8.93 | 3.38 | 0 | 5.86 | 65.38 | 65.38 |
| BCRC14535 | | | | | | |
| BCRC14535 | 6.21 | 1.96 | 0 | 3.72 | 62.09 | 62.09 |

As shown in Table 11, as compared with the system comprising Clostridium tyrobutyricum BCRC 14535 alone, the system comprising both Clostridium Clostridium cadaveris BCRC 14511 and Clostridium tyrobutyricum BCRC 14535 was much better in the consumption rates of glucose (i.e., substrate) and lactic acid (i.e., co-substrate), the production rate of organic acid, and the carbon conversion rates of butyric acid and organic acid. The above results also indicate that the Clostridium cadaveris BCRC 14511 and Clostridium tyrobutyricum BCRC 14535 co-culture system could provide better utilization rates of substrate and co-substrate, a better yield of fermentation product, and a better carbon conversion rate.

### Test 2-3-6

A CSL-CGM medium with a CSL concentration of about 15% (pH=6.0) was prepared, and then the medium was deoxygenated. Thereafter, an air-tight serum bottle was injected with 60 ml of the above deoxygenated CSL-CGM medium.

Each of the pre-cultured Clostridium cadaveris BCRC 14511 and Clostridium tyrobutyricum BCRC 14535 prepared in the Experiment 2.2 was inoculated into the above air-tight serum bottle at about 30% inoculation rate. The air-tight serum bottle was then kept in an anaerobic incubator at 37° C. and sample was taken therefrom at 24 hours. The sample was analyzed by Agilent 1100 HPLC analysis in combination with Aminex HPX-87H (300×7.8 mm) column so as to calculate the consumption of lactic acid, and the amounts of acetic acid and butyric acid in the above culture medium. In addition, the carbon conversion rates of butyric acid and organic acid were calculated. The results are shown in Table 12.

TABLE 12

| | | | | Carbon | Carbon |
|---|---|---|---|---|---|
| | Consumption | Amount | Amount | conversion | conversion |
| | of lactic acid | of acetic | of butyric | rate of butyric | rate of organic |
| Strain | (g/L) | acid (g/L) | acid (g/L) | acid (%) | acid (%) |
| BCRC14511 + | 11.97 | 0 | 8.38 | 95.46 | 95.46 |
| BCRC14535 | | | | | |

As shown in Table 12, for the medium using the system comprising both Clostridium cadaveris BCRC 14511 and Clostridium tyrobutyricum BCRC 14535, even though only CSL (which contained protein, lactic acid, and minor saccharide) but not glucose (i.e., substrate) was added thereto, production of butyric acid could also be detected and the carbon conversion rates of butyric acid and organic acid both reached 95.46% (much higher than the traditional theoretical value of 66%). The result indicates that the Clostridium cadaveris BCRC 14511 and Clostridium tyrobutyricum BCRC 14535 co-culture system could convert an amino acid or lactic acid into product such as butyric acid under a condition without glucose, and it could provide a good carbon conversion rate.

### Test 2-3-7

CGM medium was mixed with xylose and lactate to provide a medium mixture with a xylose concentration of 2 g/L and a lactic acid concentration of 5 g/L (pH=6.0), and then the medium mixture was deoxygenated. Each of two air-tight serum bottles was injected with 60 ml of the above deoxygenated medium mixture.

Each of the pre-cultured Clostridium sporogenes BCRC 11259 and Clostridium tyrobutyricum BCRC 14535 prepared in the Experiment 2.2 was inoculated into one of the above two air-tight serum bottles at about 30% inoculation rate; and the pre-cultured Clostridium tyrobutyricum BCRC 14535 prepared in the Experiment 2.2 was inoculated into the other air-tight serum bottle at about 30% inoculation rate. The two air-tight serum bottles were then kept in an anaerobic incubator at 37° C. and samples were taken therefrom at 30 hours. The samples were analyzed by Agilent 1100 HPLC analysis in combination with Aminex HPX-87H (300×7.8 mm) column so as to calculate the consumptions of xylose and lactic acid, the amounts of acetic acid and butyric acid in the above culture medium. In addition, the carbon conversion rates of butyric acid and organic acid were calculated. The results are shown in Table 13.

TABLE 13

| | | | | | Carbon | Carbon |
|---|---|---|---|---|---|---|
| | | | | | conversion | conversion |
| | Consumption | Consumption | Amount of | Amount of | rate of | rate of |
| | of xylose | of lactic acid | acetic acid | butyric | butyric | organic |
| Strain | (g/L) | (g/L) | (g/L) | acid (g/L) | acid (%) | acid (%) |
| BCRC11259 + | 1.9 | 4.9 | 0.1 | 3.3 | 66.18 | 67.65 |
| BCRC14535 | | | | | | |
| BCRC14535 | 0.3 | 0 | 0 | 0.1 | 45.45 | 45.45 |

As shown in Table 13, as compared with the system comprising Clostridium tyrobutyricum BCRC 14535 alone, the system comprising both Clostridium sporogenes BCRC 11259 and Clostridium tyrobutyricum BCRC 14535 was much better in the consumption rates of xylose (i.e., substrate) and lactic acid (i.e., co-substrate), the production rate of organic acid, and the carbon conversion rates of butyric acid and organic acid. The above results also indicate that the Clostridium sporogenes BCRC 11259 and Clostridium tyrobutyricum BCRC 14535 co-culture system could provide better utilization rates of substrate and co-substrate, a better yield of fermentation product, and a better carbon conversion rate.

### Example 3

### Use of a Microorganism Co-Culture System Containing a First Strain, a Second Strain and a Third Strain in the Production of an Organic Acid or an Alcohol

### Experiment 3-1: Strains

In example 3, one of Clostridium ljungdahlii BCRC 17797, Terrisporobacter glycolicus BCRC 14553, and Clostridium scatologenes BCRC 14540, all are able to fix carbon oxide, was used as the first strain. Clostridium cadaveris BCRC 14511, which is able to fermentatively metabolize amino acid, was used as the second strain. And one of Clostridium tyrobutyricum BCRC 14535 and Clostridium beijerinckii BCRC 14488, both are able to metabolize saccharide ororganic compound to produce organic acid or alcohol (such as acetic acid, butyric acid, and butanol) in fermentation, was used as the third strain.

### 3-2. Pre-Culture

(a) Clostridium ljungdahlii BCRC 17797: a single colony of this strain was selected, inoculated in 10 ml deoxygenated RCM medium being externally added with 10 g/L fructose, and incubated in an anaerobic incubator at 37° C. for 48 hours so as to let the OD₆₀₀ (the absorbance at a wavelength of 600 nm) of the strain reach about 1.0 to 1.2.
(b) Terrisporobacter glycolicus BCRC 14553/Clostridium scatologenes BCRC 14540/Clostridium cadaveris BCRC 14511/Clostridium tyrobutyricum BCRC 14535/Clostridium beijerinckii BCRC 14488: a single colony of the strain was selected, inoculated in 10 ml deoxygenated RCM medium, and incubated in an anaerobic incubator at 37° C. for 14 to 16 hours so as to let the OD₆₀₀ (the absorbance at a wavelength of 600 nm) of the strain reach about 1.0 to 1.2.

### Experiment 3-3: Fermentation Tests

### Test 3-3-1

CGM medium was mixed with glucose and lactate to provide a medium mixture with a glucose concentration of 5 g/L and a lactic acid concentration of 5 g/L (pH=6.0), and then the medium mixture was deoxygenated. Each of two air-tight serum bottles was injected with 60 ml of the above deoxygenated medium mixture.

Each of the pre-cultured Clostridium ljungdahlii BCRC 17797, Clostridium cadaveris BCRC 14511, and Clostridium tyrobutyricum BCRC 14535 prepared in the Experiment 3.2 were inoculated into one of the above two air-tight serum bottles at about 30% inoculation rate; and the pre-cultured Clostridium tyrobutyricum BCRC 14535 prepared in the Experiment 3.2 was inoculated into the other air-tight serum bottle at about 30% inoculation rate. The two air-tight serum bottles were then kept in an anaerobic incubator at 37° C. and samples were taken therefrom at 24 hours. The samples were analyzed by Agilent 1100 HPLC analysis in combination with Aminex HPX-87H (300×7.8 mm) column so as to calculate the consumptions of glucose and lactic acid, the amounts of acetic acid and butyric acid in the above culture medium. In addition, the carbon conversion rates of butyric acid and organic acid were calculated. The results are shown in Table 14.

TABLE 14

| | | | | | Carbon | Carbon |
|---|---|---|---|---|---|---|
| | | | | | conversion | conversion |
| | Consumption | Consumption | Amount of | Amount of | rate of | rate of |
| | of glucose | of lactic acid | acetic acid | butyric | butyric | organic |
| Strain | (g/L) | (g/L) | (g/L) | acid (g/L) | acid (%) | acid (%) |
| BCRC17797 + | 4.83 | 5.17 | 0.42 | 5.76 | 78.56 | 82.76 |
| BCRC14511 + | | | | | | |
| BCRC14535 | | | | | | |
| BCRC14535 | 1.46 | 0.08 | 0 | 0.49 | 42.72 | 42.72 |

As shown in Table 14, as compared with the system comprising Clostridium tyrobutyricum BCRC 14535 alone, which provided a poor consumption rate of glucose (i.e., substrate) and hardly metabolized lactic acid (i.e., co-substrate), the system comprising Clostridium ljungdahlii BCRC 17797, Clostridium cadaveris BCRC 14511, and Clostridium tyrobutyricum BCRC 14535 was much better in the consumption rates of glucose and lactic acid, the production rate of organic acid, and the carbon conversion rates of butyric acid and organic acid. The above results also indicate that the Clostridium ljungdahlii BCRC 17797, Clostridium cadaveris BCRC 14511, and Clostridium tyrobutyricum BCRC 14535 co-culture system is better in the utilization rates of substrate and co-substrate, the yield of fermentation product, and the carbon conversion rate, and its carbon conversion rate is much higher than the traditional theoretical value (i.e., 66%).

### Test 3-3-2

CGM medium was mixed with glucose to provide a medium mixture with a glucose concentration of 10 g/L (pH=6.0), and then the medium mixture was deoxygenated. Each of three air-tight serum bottles was injected with 60 ml of the above deoxygenated medium mixture.

Each of the pre-cultured Clostridium ljungdahlii BCRC 17797, Clostridium cadaveris BCRC 14511, and Clostridium tyrobutyricum BCRC 14535 prepared in Experiment 3.2 was inoculated into the first air-tight serum bottle of the above three bottles at about 30% inoculation rate; each of the pre-cultured Clostridium ljungdahlii BCRC 17797 and Clostridium tyrobutyricum BCRC 14535 was inoculated into the second air-tight serum bottle at about 30% inoculation rate; and each of the pre-cultured Clostridium cadaveris BCRC 14511 and Clostridium tyrobutyricum BCRC 14535 was inoculated into the third air-tight serum bottle at about 30% inoculation rate. The three air-tight serum bottles were then kept in an anaerobic incubator at 37° C. and samples were taken therefrom after incubating for 7 hours. The samples were analyzed by Agilent 1100 HPLC analysis in combination with Aminex HPX-87H (300×7.8 mm) column so as to calculate the consumption of glucose, the amounts of acetic acid and butyric acid in the above culture medium. In addition, the carbon conversion rates of butyric acid and organic acid were calculated. The results are shown in Table 15.

TABLE 15

| | | | | | Carbon | Carbon |
|---|---|---|---|---|---|---|
| | | | | | conversion | conversion |
| | | Consumption | Amount | Amount of | rate of | rate of |
| | | of glucose | of acetic | butyric | butyric | organic |
| Group | Strain | (g/L) | acid (g/L) | acid (g/L) | acid (%) | acid (%) |
| 1 | BCRC17797 + | 6.01 | 0.77 | 2.76 | 64.24 | 77.36 |
| | BCRC14511 + | | | | | |
| | BCRC14535 | | | | | |
| 2 | BCRC17797 + | 5.03 | 0.60 | 2.13 | 57.59 | 69.53 |
| | BCRC14535 | | | | | |
| 3 | BCRC14511 + | 6.02 | 0.33 | 2.55 | 57.90 | 63.37 |
| | BCRC14535 | | | | | |

As shown in Table 15, as compared with the group 2 or group 3 microorganism co-culture system, the group 1 microorganism co-culture system was much better in the production rate of organic acid and the carbon conversion rates of butyric acid and organic acid. The above results indicate that as compared with the co-culture system comprising two strains, the co-culture system with Clostridium ljungdahlii BCRC 17797, Clostridium cadaveris BCRC 14511, and Clostridium tyrobutyricum BCRC 14535 could provide a much better yield of fermentation product and a better carbon conversion rate.

### Test 3-3-3

CGM medium was mixed with lactate to provide a medium mixture with a lactic acid concentration of 10 g/L (pH=6.0), and then the medium mixture was deoxygenated. Each of two air-tight serum bottles was injected with 60 ml of the above deoxygenated medium mixture.

Each of the pre-cultured Clostridium ljungdahlii BCRC 17797, Clostridium cadaveris BCRC 14511, and Clostridium tyrobutyricum BCRC 14535 prepared in the Experiment 3.2 was inoculated into one of the above two air-tight serum bottles at about 30% inoculation rate; and the pre-cultured Clostridium tyrobutyricum BCRC 14535 prepared in the Experiment 3.2 was inoculated into the other air-tight serum bottle at about 30% inoculation rate. The two air-tight serum bottles were then kept in an anaerobic incubator at 37° C. and samples were taken therefrom at 24 hours. The samples were analyzed by Agilent 1100 HPLC analysis in combination with Aminex HPX-87H (300×7.8 mm) column so as to calculate the consumption of lactic acid, and amounts of acetic acid and butyric acid in the above culture medium. The results are shown in Table 16.

TABLE 16

| | | Amount of | Amount of |
|---|---|---|---|
| | Consumption of | acetic acid | butyric acid |
| Strain | lactic acid (g/L) | (g/L) | (g/L) |
| BCRC17797 + | 6.49 | 0.32 | 5.55 |
| BCRC14511 + | | | |
| BCRC14535 | | | |
| BCRC14535 | 0.14 | 0 | 0 |

As shown in Table 16, as compared with the system comprising Clostridium tyrobutyricum BCRC 14535 alone, which hardly metabolized lactic acid, the system comprising Clostridium ljungdahlii BCRC 17797, Clostridium cadaveris BCRC 14511, and Clostridium tyrobutyricum BCRC 14535 could metabolize lactic acid efficiently and produce organic acid. The above results indicate that the co-culture system with Clostridium ljungdahlii BCRC 17797, Clostridium cadaveris BCRC 14511, and Clostridium tyrobutyricum BCRC 14535 could convert lactic acid into product such as acetic acid and butyric acid under a condition without glucose, and provide a good yield of fermentation product.

### Test 3-3-4

CSL-CGM medium was mixed with glucose to provide a medium mixture with a glucose concentration of 12 g/L and a CSL concentration of about 3.5% (pH=6.0), and then the medium mixture was deoxygenated. Each of three air-tight serum bottles was injected with 60 ml of the above deoxygenated medium mixture.

Each of the pre-cultured Clostridium ljungdahlii BCRC 17797, Clostridium cadaveris BCRC 14511, and Clostridium tyrobutyricum BCRC 14535 prepared in the Experiment 3.2 was inoculated into the first air-tight serum bottle of the above three bottles at about 30% inoculation rate; each of the pre-cultured Clostridium ljungdahlii BCRC 17797 and Clostridium tyrobutyricum BCRC 14535 prepared in the Experiment 3.2 was inoculated into the second air-tight serum bottle at about 30% inoculation rate; and each of the pre-cultured Clostridium cadaveris BCRC 14511 and Clostridium tyrobutyricum BCRC 14535 prepared in the Experiment 3.2 was inoculated into the third air-tight serum bottle at about 30% inoculation rate. The three air-tight serum bottles were then kept in an anaerobic incubator at 37° C. and samples were taken therefrom at 24 hours. The samples were analyzed by Agilent 1100 HPLC analysis in combination with Aminex HPX-87H (300×7.8 mm) column so as to calculate the consumptions of glucose and lactic acid, the amounts of acetic acid and butyric acid in the above culture medium. In addition, the carbon conversion rates of butyric acid and organic acid were calculated. The results are shown in Table 17.

TABLE 17

| | | | | | Amount | Carbon | Carbon |
|---|---|---|---|---|---|---|---|
| | | | | Amount | of | conversion | conversion |
| | | Consumption | Consumption | of acetic | butyric | rate of | rate of |
| | | of glucose | of lactic acid | acid | acid | butyric | organic |
| Group | Strain | (g/L) | (g/L) | (g/L) | (g/L) | acid (%) | acid (%) |
| 1 | BCRC17797 + | 12.05 | 3.30 | 0.46 | 7.99 | 71.01 | 74.01 |
| | BCRC14511 + | | | | | | |
| | BCRC14535 | | | | | | |
| 2 | BCRC17797 + | 12.15 | 3.29 | 0.59 | 7.76 | 68.57 | 72.39 |
| | BCRC14535 | | | | | | |
| 3 | BCRC14511 + | 9.7 | 2.65 | 0 | 5.8 | 64.04 | 64.04 |
| | BCRC14535 | | | | | | |

As shown in Table 17, as compared with the group 2 or group 3 microorganism co-culture system, the group 1 microorganism co-culture system was much better in the carbon conversion rates of butyric acid and organic acid. The above results indicate that as compared with the co-culture system comprising two strains, the co-culture system with Clostridium ljungdahlii BCRC 17797, Clostridium cadaveris BCRC 14511, and Clostridium tyrobutyricum BCRC 14535 could provide a much better carbon conversion rate.

### Test 3-3-5

CSL-CGM medium was mixed with glucose to provide a medium mixture with a glucose concentration of 10 g/L and a CSL concentration of about 5% (pH=6.0), and then the medium mixture was deoxygenated. Each of three air-tight serum bottles was injected with 60 ml of the above deoxygenated medium mixture.

Each of the pre-cultured Clostridium ljungdahlii BCRC 17797, Clostridium cadaveris BCRC 14511, and Clostridium tyrobutyricum BCRC 14535 prepared in the Experiment 3.2 was inoculated into the first air-tight serum bottle of the above three bottles at about 30% inoculation rate; each of the pre-cultured Clostridium ljungdahlii BCRC 17797 and Clostridium tyrobutyricum BCRC 14535 prepared in the Experiment 3.2 was inoculated into the second air-tight serum bottle at about 30% inoculation rate; and each of the pre-cultured Clostridium cadaveris BCRC 14511 and Clostridium tyrobutyricum BCRC 14535 prepared in the Experiment 3.2 was inoculated into the third air-tight serum bottle at about 30% inoculation rate. The three air-tight serum bottles were then kept in an anaerobic incubator at 37° C. and samples were taken therefrom at 24 hours. The samples were analyzed by Agilent 1100 HPLC analysis in combination with Aminex HPX-87H (300×7.8 mm) column so as to calculate the consumptions of glucose and lactic acid, the amounts of acetic acid and butyric acid in the above culture medium. In addition, the carbon conversion rates of butyric acid and organic acid were calculated. The results are shown in Table 18.

TABLE 18

| | | | | | | Carbon | Carbon |
|---|---|---|---|---|---|---|---|
| | | | | | | conversion | conversion |
| | | Consumption | Consumption | Amount | Amount | rate of | rate of |
| | | of glucose | of lactic acid | of acetic | of butyric | butyric | organic |
| Group | Strain | (g/L) | (g/L) | acid (g/L) | acid (g/L) | acid (%) | acid (%) |
| 1 | BCRC17797 + | 10.11 | 5.02 | 0.83 | 8.15 | 73.45 | 78.94 |
| | BCRC14511 + | | | | | | |
| | BCRC14535 | | | | | | |
| 2 | BCRC17797 + | 10.2 | 5.2 | 0.98 | 7.77 | 69.62 | 76.05 |
| | BCRC14535 | | | | | | |
| 3 | BCRC14511 + | 10.11 | 3.27 | 0 | 6.36 | 64.82 | 64.82 |
| | BCRC14535 | | | | | | |

As shown in Table 18, as compared with the group 2 or group 3 microorganism co-culture system, the group 1 microorganism co-culture system was much better in the production rate of butyric acid, and the carbon conversion rates of butyric acid and organic acid. The above results indicate that as compared with the co-culture system comprising two strains, the co-culture system with Clostridium ljungdahlii BCRC 17797, Clostridium cadaveris BCRC 14511, and Clostridium tyrobutyricum BCRC 14535 could provide a better yield of butyric acid and a better carbon conversion rate.

### Test 3-3-6

CSL-CGM medium was mixed with glucose to provide a medium mixture with a glucose concentration of 10 g/L and a CSL concentration of about 7% (pH=6.0), and then the medium mixture was deoxygenated. Each of three air-tight serum bottles was injected with 60 ml of the above deoxygenated medium mixture.

Each of the pre-cultured Clostridium ljungdahlii BCRC 17797, Clostridium cadaveris BCRC 14511, and Clostridium tyrobutyricum BCRC 14535 prepared in the Experiment 3.2 was inoculated into the first air-tight serum bottle of the above three bottles at about 30% inoculation rate; each of the pre-cultured Clostridium ljungdahlii BCRC 17797 and Clostridium tyrobutyricum BCRC 14535 prepared in the Experiment 3.2 was inoculated into the second air-tight serum bottle at about 30% inoculation rate; and each of the pre-cultured Clostridium cadaveris BCRC 14511 and Clostridium tyrobutyricum BCRC 14535 prepared in the Experiment 3.2 was inoculated into the third air-tight serum bottle at about 30% inoculation rate. The three air-tight serum bottles were then kept in an anaerobic incubator at 37° C. and samples were taken therefrom at 24 hours. The samples were analyzed by Agilent 1100 HPLC analysis in combination with Aminex HPX-87H (300×7.8 mm) column so as to calculate the consumptions of glucose and lactic acid, the amounts of acetic acid and butyric acid in the above culture medium. In addition, the carbon conversion rates of butyric acid and organic acid were calculated. The results are shown in Table 19.

TABLE 19

| | | | | | | Carbon | Carbon |
|---|---|---|---|---|---|---|---|
| | | | | | | conversion | conversion |
| | | Consumption | Consumption | Amount | Amount | rate of | rate of |
| | | of glucose | of lactic acid | of acetic | of butyric | butyric | organic |
| Group | Strain | (g/L) | (g/L) | acid (g/L) | acid (g/L) | acid (%) | acid (%) |
| 1 | BCRC17797 + | 9.12 | 7.05 | 0.83 | 9.43 | 79.56 | 84.69 |
| | BCRC14511 + | | | | | | |
| | BCRC14535 | | | | | | |
| 2 | BCRC17797 + | 9.34 | 7.11 | 1.1 | 9.01 | 74.69 | 81.38 |
| | BCRC14535 | | | | | | |
| 3 | BCRC14511 + | 8.93 | 3.38 | 0 | 5.86 | 65.38 | 65.38 |
| | BCRC14535 | | | | | | |

As shown in Table 19, as compared with the group 2 or group 3 microorganism co-culture system, the group 1 microorganism co-culture system was much better in the production rate of butyric acid, and the carbon conversion rates of butyric acid and organic acid. The above results indicate that as compared with the co-culture system comprising two strains, the co-culture system with Clostridium ljungdahlii BCRC 17797, Clostridium cadaveris BCRC 14511, and Clostridium tyrobutyricum BCRC 14535 could provide a better yield of butyric acid and a better carbon conversion rate.

### Test 3-3-7

A CSL-CGM medium with a CSL concentration of about 15% (pH=6.0) was prepared, and then the medium was deoxygenated. Each of three air-tight serum bottles was injected with 60 ml of the above deoxygenated CSL-CGM medium.

Each of the pre-cultured Clostridium ljungdahlii BCRC 17797, Clostridium cadaveris BCRC 14511, and Clostridium tyrobutyricum BCRC 14535 prepared in the Experiment 3.2 was inoculated into the first air-tight serum bottle of the above three bottles at about 30% inoculation rate; each of the pre-cultured Clostridium ljungdahlii BCRC 17797 and Clostridium tyrobutyricum BCRC 14535 prepared in the Experiment 3.2 was inoculated into the second air-tight serum bottle at about 30% inoculation rate; and each of the pre-cultured Clostridium cadaveris BCRC 14511 and Clostridium tyrobutyricum BCRC 14535 prepared in the Experiment 3.2 was inoculated into the third air-tight serum bottle at about 30% inoculation rate. The three air-tight serum bottles were then kept in an anaerobic incubator at 37° C. and samples were taken therefrom at 24 hours. The samples were analyzed by Agilent 1100 HPLC analysis in combination with Aminex HPX-87H (300×7.8 mm) column so as to calculate the consumption of lactic acid and the amount of butyric acid in the above culture medium. The results are shown in Table 20.

TABLE 20

| | | Consumption | Amount of |
|---|---|---|---|
| | | of lactic acid | butyric acid |
| Group | Strain | (g/L) | (g/L) |
| 1 | BCRC17797 + | 10.24 | 9.49 |
| | BCRC14511 + | | |
| | BCRC14535 | | |
| 2 | BCRC17797 + | 13.57 | 9.11 |
| | BCRC14535 | | |
| 3 | BCRC14511 + | 11.97 | 8.38 |
| | BCRC14535 | | |

As shown in Table 20, as compared with the group 2 or group 3 microorganism co-culture system, the group 1 microorganism co-culture system could provide a lower consumption rate of lactic acid and a higher production rate of butyric acid. The above results indicate that as compared with the co-culture system comprising two strains, the co-culture system with Clostridium ljungdahlii BCRC 17797, Clostridium cadaveris BCRC 14511, and Clostridium tyrobutyricum BCRC 14535 could provide a less consumption of lactic acid, and a better production rate of butyric acid.

### Test 3-3-8

CGM medium was mixed with lactate to provide a medium mixture with a lactic acid concentration of 15 g/L (pH=6.0), and then the medium mixture was deoxygenated. Thereafter, an air-tight serum bottle was injected with 50 ml of the above deoxygenated medium mixture.

Each of the pre-cultured Terrisporobacter glycolicus BCRC 14553, Clostridium cadaveris BCRC 14511, and Clostridium tyrobutyricum BCRC 14535 prepared in the Experiment 3.2 was inoculated into the above air-tight serum bottle at about 20% inoculation rate. The air-tight serum bottle was then kept in an anaerobic incubator at 37° C. and sample was taken therefrom at 43 hours. The sample was analyzed by Agilent 1100 HPLC analysis in combination with Aminex HPX-87H (300×7.8 mm) column so as to calculate the consumption of lactic acid and the amounts of acetic acid and butyric acid in the above culture medium. In addition, the carbon conversion rates of butyric acid and organic acid were calculated. The results are shown in Table 21.

TABLE 21

| | | | | Carbon | Carbon |
|---|---|---|---|---|---|
| | Consumption | Amount of | Amount of | conversion rate | conversion rate |
| | of lactic acid | acetic acid | butyric acid | of butyric acid | of organic acid |
| Strain | (g/L) | (g/L) | (g/L) | (%) | (%) |
| BCRC14553 + | 14.41 | 0 | 9.34 | 88.34 | 88.34 |
| BCRC14511 + | | | | | |
| BCRC14535 | | | | | |

As shown in Table 21, the co-culture system with Terrisporobacter glycolicus BCRC 14553, Clostridium cadaveris BCRC 14511, and Clostridium tyrobutyricum BCRC 14535 could provide a carbon conversion rate of 88.34% (much higher than the traditional theoretical value of 66%).

### Test 3-3-9

CSL-CGM medium was mixed with lactate to provide a medium mixture with a lactic acid concentration of 20 g/L and a CSL concentration of about 5% (pH=6.0), and then the medium mixture was deoxygenated. Thereafter, an air-tight serum bottle was injected with 50 ml of the above deoxygenated medium mixture.

Each of the pre-cultured Terrisporobacter glycolicus BCRC 14553, Clostridium cadaveris BCRC 14511, and Clostridium tyrobutyricum BCRC 14535 prepared in the Experiment 3.2 was inoculated into the above air-tight serum bottle at about 20% inoculation rate. The air-tight serum bottle was then kept in an anaerobic incubator at 37° C. and sample was taken therefrom at 65 hours. The sample was analyzed by Agilent 1100 HPLC analysis in combination with Aminex HPX-87H (300×7.8 mm) column so as to calculate the consumption of lactic acid and the amounts of acetic acid and butyric acid in the above culture medium. In addition, the carbon conversion rate of butyric acid was calculated. The results are shown in Table 22.

TABLE 22

| | | | | Carbon |
|---|---|---|---|---|
| | Consumption | Amount of | Amount of | conversion rate |
| | of lactic acid | acetic acid | butyric acid | of butyric acid |
| Strain | (g/L) | (g/L) | (g/L) | (%) |
| BCRC14553 + | 18.4 | 4 | 13.1 | 98 |
| BCRC14511 + | | | | |
| BCRC14535 | | | | |

As shown in Table 22, the co-culture system with Terrisporobacter glycolicus BCRC 14553, Clostridium cadaveris BCRC 14511, and Clostridium tyrobutyricum BCRC 14535 could consume 18.4 g/L of lactic acid, and produce 13.1 g/L of butyric acid, had a carbon conversion rate of 98% (much higher than the traditional theoretical value of 66%), and could produce additional 4 g/L of acetic acid.

### Test 3-3-10

mPETC medium was mixed with lactate to provide a medium mixture with a lactic acid concentration of 6 g/L (pH=6.0), and then the medium mixture was deoxygenated. Each of two air-tight serum bottles was injected with 50 ml of the above deoxygenated medium mixture.

Each of the pre-cultured Terrisporobacter glycolicus BCRC 14553, Clostridium cadaveris BCRC 14511, and Clostridium tyrobutyricum BCRC 14535 prepared in Experiment 3.2 were inoculated into one of the above air-tight serum bottle at about 20% inoculation rate, and at the presence of 20 psi of externally introduced syngas (20% carbon dioxide, 80% hydrogen) as a gaseous co-substrate (hereinafter referred to as the “with syngas” experimental group). The above strain inoculation steps were repeated while in the absence of any externally introduced gas (hereinafter referred to as the “without additional gas” control group). The two air-tight serum bottles were then kept in an anaerobic incubator at 37° C. and samples were taken therefrom at 48 hours. The sample was analyzed by Agilent 1100 HPLC analysis in combination with Aminex HPX-87H (300×7.8 mm) column so as to calculate the consumption of lactic acid and the amounts of acetic acid and butyric acid in the above culture medium. In addition, the carbon conversion rate of butyric acid was calculated. The results are shown in Table 23.

TABLE 23

| | | | | | Carbon |
|---|---|---|---|---|---|
| | | Consumption | Amount of | Amount of | conversion |
| | | of lactic acid | acetic acid | butyric acid | rate of butyric |
| Group | Strain | (g/L) | (g/L) | (g/L) | acid (%) |
| “with syngas” | BCRC14553 + | 6.2 | 3.3 | 4.5 | 98.97 |
| experimental | BCRC14511 + | | | | |
| group | BCRC14535 | | | | |
| “without | BCRC14553 + | 6.2 | 1.3 | 4.5 | 98.97 |
| additional | BCRC14511 + | | | | |
| gas” control | BCRC14535 | | | | |
| group | | | | | |

As shown in Table 23, as compared with the microorganism co-culture system of the “without additional gas” control group, the amount of acetic acid provided by the microorganism co-culture system of the “with syngas” experimental group was markedly increased. The result indicates that the co-culture system of Terrisporobacter glycolicus BCRC 14553, Clostridium cadaveris BCRC 14511, and Clostridium tyrobutyricum BCRC 14535 could use syngas (carbon dioxide and hydrogen) efficiently to produce more acetic acid, and provide a better output of fermentation product.

### Test 3-3-11

The steps of test 3-3-10 were repeated, but the Terrisporobacter glycolicus BCRC 14553 was replaced with pre-cultured Clostridium scatologenes BCRC 14540 prepared in Experiment 3-2. The results are shown in Table 24.

TABLE 24

| | | | | | Carbon |
|---|---|---|---|---|---|
| | | Consumption | Amount of | Amount of | conversion |
| | | of lactic acid | acetic acid | butyric acid | rate of butyric |
| Group | Strain | (g/L) | (g/L) | (g/L) | acid (%) |
| “with syngas” | BCRC14540 + | 5.7 | 3.8 | 3.6 | 86.12 |
| experimental | BCRC14511 + | | | | |
| group | BCRC14535 | | | | |
| “without | BCRC14540 + | 5.7 | 2.2 | 3.6 | 86.12 |
| additional | BCRC14511 + | | | | |
| gas” control | BCRC14535 | | | | |
| group | | | | | |

As shown in Table 24, as compared with the microorganism co-culture system of the “without additional gas” control group, the amount of acetic acid provided by the microorganism co-culture system of the “with syngas” experimental group was markedly increased. The result indicates that the co-culture system with Clostridium scatologenes BCRC 14540, Clostridium cadaveris BCRC 14511, and Clostridium tyrobutyricum BCRC 14535 could use syngas (carbon dioxide and hydrogen) efficiently to produce more acetic acid, and provide a better output of fermentation product.

### Test 3-3-12

P2 medium was mixed with glucose to provide a medium mixture with a glucose concentration of 20 g/L (pH=6.0), and then the medium mixture was deoxygenated. Thereafter, an air-tight serum bottle was injected with 50 ml of the above deoxygenated medium mixture.

Each of the pre-cultured Clostridium ljungdahlii BCRC 17797, Clostridium cadaveris BCRC 14511, and Clostridium beijerinckii BCRC 14488 prepared in the Experiment 3.2 was inoculated into the above air-tight serum bottle at about 20% inoculation rate. The air-tight serum bottle was then kept in an anaerobic incubator at 37° C. and sample was taken therefrom at 96 hours. The sample was analyzed by Agilent 1100 HPLC analysis in combination with Aminex HPX-87H (300×7.8 mm) column so as to calculate the consumption of glucose and the amounts of acetic acid, butyric acid, and butanol in the above culture medium. In addition, the carbon conversion rate of total products was calculated. The results are shown in Table 25.

TABLE 25

| | | | | | Carbon |
|---|---|---|---|---|---|
| | Consumption | Amount | Amount of | | conversion |
| | of glucose | of acetic acid | butyric acid | Amount of | rate of total |
| Strain | (g/L) | (g/L) | (g/L) | butanol (g/L) | products (%) |
| BCRC17797 + | 17.5 | 5.5 | 5.6 | 1.2 | 86.18 |
| BCRC14511 + | | | | | |
| BCRC14488 | | | | | |

As shown in Table 25, the system comprising Clostridium beijerinckii BCRC 14488 (which is able to produce alcohol), Clostridium ljungdahlii BCRC 17797, and Clostridium cadaveris BCRC 14511 could perform fermentation under an anaerobic condition to produce organic acid and alcohol, and the carbon conversion rate of total products thereof could reach 86.18%, which is much higher than the traditional theoretical value (i.e., 66%).

### 3-4. Test of Stability

### Test 3-4-1

CGM medium was mixed with lactate to provide a medium mixture with a lactic acid concentration of 20 g/L (pH=6.0), and then the medium mixture was deoxygenated. Thereafter, an air-tight serum bottle was injected with 50 ml of the above deoxygenated medium mixture.

A first batch fermentation was performed by the following steps: each of the pre-cultured Terrisporobacter glycolicus BCRC 14553, Clostridium cadaveris BCRC 14511, and Clostridium tyrobutyricum BCRC 14535 prepared in Experiment 3.2 was inoculated into the above air-tight serum bottle at about 20% inoculation rate. The air-tight serum bottle was then kept in an anaerobic incubator at 37° C. and sample was taken therefrom at 24 hours. The sample was analyzed by Agilent 1100 HPLC analysis in combination with Aminex HPX-87H (300×7.8 mm) column.

Thereafter, a second batch fermentation was performed by the following steps: 40 ml of strain liquid was taken from the air-tight serum bottle and centrifuged (6000 g, 10 minutes), and then the strains were collected. The collected strains were re-suspended in and washed by CGM medium, then the medium was centrifuged (6000 g, 10 minutes). The strains were re-cultured into the above deoxygenated medium mixture with a lactic acid concentration of 20 g/L, and kept in an anaerobic incubator at 37° C. and sample was taken therefrom at 24 hours. The sample was analyzed by Agilent 1100 HPLC analysis in combination with Aminex HPX-87H (300×7.8 mm) column.

The consumption of lactic acid and the amounts of acetic acid and butyric acid in the fermentation medium of the first batch and second batch were calculated. In addition, the carbon conversion rates of butyric acid and organic acid were calculated. The results are shown in Table 26.

TABLE 26

| | | | | | Carbon | Carbon |
|---|---|---|---|---|---|---|
| | | | | | conversion | conversion |
| | | Consumption | Amount of | Amount of | rate of | rate of |
| | | of lactic acid | acetic acid | butyric acid | butyric | organic |
| Batch | Strain | (g/L) | (g/L) | (g/L) | acid (%) | acid (%) |
| 1 | BCRC14553 + | 19.3 | 0.2 | 11.7 | 82.67 | 83.7 |
| | BCRC14511 + | | | | | |
| | BCRC14535 | | | | | |
| 2 | BCRC14553 + | 19.2 | 0.2 | 11.5 | 81.68 | 82.72 |
| | BCRC14511 + | | | | | |
| | BCRC14535 | | | | | |

As shown in Table 26, the consumption of lactic acid, the amounts of acetic acid and butyric acid, and the carbon conversion rate of butyric acid in the second batch fermentation were almost the same as those in the first batch fermentation. The results indicate that the microorganism co-culture system in accordance with the present invention could maintain stable microflora and stable interaction among microorganism strains.

### Test 3-4-2

Steps of test 3-4-1 were repeated, but the medium mixture with a lactic acid concentration of 20 g/L was replaced by CSL-CGM with a CSL concentration of about 25% (pH=6.0). The results are shown in Table 27.

TABLE 27

| | | | | Amount |
|---|---|---|---|---|
| | | Consumption of | Amount of acetic | of butyric |
| Batch | Strain | lactic acid (g/L) | acid (g/L) | acid (g/L) |
| 1 | BCRC14553 + | 19.7 | 2.2 | 16 |
| | BCRC14511 + | | | |
| | BCRC14535 | | | |
| 2 | BCRC14553 + | 19.4 | 1.9 | 15.5 |
| | BCRC14511 + | | | |
| | BCRC14535 | | | |

As shown in Table 27, the consumption of lactic acid, and the amounts of acetic acid and butyric acid in the second batch fermentation were almost the same as those in the first batch fermentation. The result indicates again that the microorganism co-culture system in accordance with the present invention could maintain stable microflora and stable interaction among microorganism strains.

### Test 3-4-3

CSL-CGM medium was mixed with glucose at different ratios to provide the following three different mediums: a CSL-CGM medium with a glucose concentration of 10 g/L and a CSL concentration of about 3.5% (pH=6.0), a CSL-CGM medium with a glucose concentration of 8 g/L and a CSL concentration of about 10% (pH=6.0), and a CSL-CGM medium with a CSL concentration of about 12% (pH=6.0). Thereafter, the three mediums were deoxygenated. Each of the three air-tight serum bottles was injected with one of the above deoxygenated medium mixture at an amount of 100 ml, respectively.

On the other hand, the pre-cultured Clostridium ljungdahlii BCRC 17797, Clostridium cadaveris BCRC 14511, and Clostridium tyrobutyricum BCRC 14535 prepared in the Experiment 3-2 were taken and well mixed, and the strain mixture thus obtained was immobilized by PVA (polyvinyl alcohol) to provide co-culture PVA particles. The co-culture PVA particles thus obtained were inoculated into the above deoxygenated mediums at about 5% inoculation amount (volume/volume). The air-tight serum bottles were then kept in an anaerobic incubator at 37° C. and samples were taken therefrom at 50 hours or 60 hours. The samples were analyzed by Agilent 1100 HPLC analysis in combination with Aminex HPX-87H (300×7.8 mm) column so as to calculate the consumptions of glucose and lactic acid, the amounts of acetic acid and butyric acid in the above culture medium. In addition, the carbon conversion rate of butyric acid was calculated. The results are shown in Table 28.

TABLE 28

| | | | | | | Carbon |
|---|---|---|---|---|---|---|
| | | | | | | conversion |
| | Incubation | Consumption | Consumption | Amount of | Amount of | rate of |
| CSL-CGM | time | of glucose | of lactic acid | acetic acid | butyric acid | butyric |
| medium mixture | (hour) | (g/L) | (g/L) | (g/L) | (g/L) | acid (%) |
| 10 g/L glucose + | 50 | 10.4 | 2.9 | 0.14 | 7.06 | 72.39 |
| 3.5% CSL | | | | | | |
| 8 g/L glucose + | 60 | 7.5 | 8.5 | 0 | 8.7 | 74.15 |
| 10% CSL | | | | | | |
| 12% CSL | 50 | — | 10.3 | 0 | 7.09 | 93.87 |

As shown in Table 28, regardless of the types of the CSL-CGM medium mixture into which the co-culture PVA particles (containing Clostridium ljungdahlii BCRC 17797, Clostridium cadaveris BCRC 14511, and Clostridium tyrobutyricum BCRC 14535 at the same time) was inoculated to perform fermentation, the carbon conversion rate of butyric acid was always higher than the traditional theoretical value (i.e., 66%).

### Test 3-4-4

A CSL-CGM medium with a CSL concentration of about 18% was prepared (pH=6.0), and then the medium was deoxygenated. An air-tight serum bottles was injected with 100 ml of the above deoxygenated medium.

On the other hand, the pre-cultured Terrisporobacter glycolicus BCRC 14553, Clostridium cadaveris BCRC 14511, and Clostridium tyrobutyricum BCRC 14535 prepared in the Experiment 3-2 were taken and well mixed, and the strain mixture thus obtained was immobilized by PVA (polyvinyl alcohol) to provide co-culture PVA particles. The co-culture PVA particles thus obtained were inoculated into the above deoxygenated medium mixture at about 5% inoculation amount (volume/volume). The air-tight serum bottle was then kept in an anaerobic incubator at 37° C. and sample was taken therefrom at 50 hours. The sample was analyzed by Agilent 1100 HPLC analysis in combination with Aminex HPX-87H (300×7.8 mm) column so as to calculate the consumption of lactic acid, the amounts of acetic acid and butyric acid in the above culture medium. In addition, the carbon conversion rate of butyric acid was calculated. The results are shown in Table 29.

TABLE 29

| | | | | | Carbon |
|---|---|---|---|---|---|
| | | | | Amount of | conversion |
| CSL- | Incubation | Consumption | Amount of | butyric | rate of |
| CGM | time | of lactic acid | acetic acid | acid | butyric |
| medium | (hour) | (g/L) | (g/L) | (g/L) | acid (%) |
| 18% CSL | 50 | 14.22 | 2.7 | 10.09 | 96.76 |

As shown in Table 29, inoculating the co-culture PVA particles (containing Terrisporobacter glycolicus BCRC 14553, Clostridium cadaveris BCRC 14511, and Clostridium tyrobutyricum BCRC 14535 at the same time) into the CSL-CGM medium with a CSL concentration of about 18% to perform fermentation, the carbon conversion rate of butyric acid was higher than the traditional theoretical value (i.e., 66%), and could produce 2.7 g/L of acetic acid.

The above results clearly indicate that the microorganism co-culture system in accordance with the present invention can maintain stable community of microorganisms and stable interaction among microorganism strains. Microorganisms in the co-culture system can live in syntrophic relationship stably, i.e., the microorganisms can interactively use the metabolites and metabolic byproducts produced in the fermentation and are in a complementary relationship (as shown in FIGS. 2A, 2B, 2C). Thus, with the use of the system in a fermentation, various feedstocks could be converted into an organic compound such as butyric acid and butanol, and the needs of using the feedstocks efficiently, reducing unnecessary carbon loss, and providing a good yield of the target product could be fulfilled.

### BRIEF DESCRIPTION OF REFERENCE NUMERALS

Not applicable.

1-20. (canceled)

21. A method of producing butyric acid, comprising:
(a) providing a microorganism co-culture system comprising an air-tight container, wherein the air-tight container includes:
(1) a substrate comprising a saccharide;
(2) at least one of a first strain and a second strain, wherein the first strain is able to fix a carbon oxide and the second strain is able to fermentatively metabolize an amino acid, and wherein the first strain produces a first metabolite in the fermentation, the second strain produces a second metabolite in the fermentation, and each of the first metabolite and the second metabolite comprises acetic acid; and
(3) a third strain, being able to metabolize the saccharide, the first metabolite and the second metabolite in the fermentation to produce butyric acid and a metabolic byproduct in fermentation, wherein the metabolic byproduct comprises carbon oxide and hydrogen,
wherein, when the second strain is present in the co-culture system, the substrate further comprises an amino acid; and
(b) keeping the microorganism co-culture system under an anaerobic atmosphere to perform the fermentation and providing a fermentation product.

22. The method according to claim 21, wherein the first strain fixes the carbon oxide of the metabolic byproduct.

23. The method according to claim 21, wherein the first strain uses the Wood-Ljungdahl (WL) pathway to fix a carbon oxide.

24. The method according to claim 23, wherein the first strain is at least one of Clostridium coskatii, Clostridium ljungdahlii, Clostridium autoethanogenum, Clostridium ragsdalei, Terrisporobacter glycolicus, and Clostridium scatologenes.

25. The method according to claim 21, wherein the second strain is at least one of Clostridium cadaveris, Clostridium sporogenes, Clostridium sticklandii, Clostridium propionicum Clostridium botulinum, and Clostridium pasteurianum.

26. The method according to claim 21, wherein the third strain is a Clostridium sp. strain.

27. The method according to claim 26, wherein the third strain is at least one of Clostridium tyrobutyricum, Clostridium butyricum, Clostridium beijerinckii, Clostridium acetobutylicum, Clostridium argentinense, Clostridium aurantibutyricum, Clostridium botulinum, Clostridium carboxidivorans, Clostridium cellulovorans, Clostridium cf. saccharolyticum, Clostridium difficile, Clostridium kluyveri, Clostridium novyi, Clostridium paraputrificum, Clostridium pascui, Clostridium peptidivorans, Clostridium perfringens, Clostridium scatologenes, Clostridium schirmacherense, Clostridium sticklandii, Clostridium subterminale S64, Clostridium symbiosum, Clostridium tetani, Clostridium tepidiprofundi Clostridium tertium, Clostridium tetanomorphum, and Clostridium thermopalmarium.

28. The method according to claim 21, wherein the microorganism co-culture system further comprises a co-substrate being lactic acid.

29. The method according to claim 28, wherein the co-substrate is a lactic acid and the saccharide and the lactic acid are used at a weight ratio of saccharide: lactic acid=1:1 to 1:10.

30. The method according to claim 21, wherein the fermentation has a carbon conversion rate of more than 66%.

31. A method of producing butanol, comprising:
(a) providing a microorganism co-culture system comprising an air-tight container, wherein the air-tight container includes:
(1) a substrate comprising a saccharide;
(2) at least one of a first strain and a second strain, wherein the first strain is able to fix a carbon oxide and the second strain is able to fermentatively metabolize an amino acid, and wherein the first strain produces a first metabolite in the fermentation, the second strain produces a second metabolite in the fermentation, and each of the first metabolite and the second metabolite comprises acetic acid; and
(3) a third strain, being able to metabolize the saccharide, the first metabolite and the second metabolite in the fermentation to produce butyric acid and/or butanol and a metabolic byproduct in fermentation, wherein the metabolic byproduct comprises carbon oxide and hydrogen,
wherein, when the second strain is present in the co-culture system, the substrate further comprises an amino acid;
(b) keeping the microorganism co-culture system under an anaerobic atmosphere to perform the fermentation and provide a fermentation product; and
(c) optionally conducting a chemical conversion reaction to convert butyric acid into butanol.

32. The method according to claim 31, wherein the first strain fixes the carbon oxide of the metabolic byproduct.

33. The method according to claim 31, wherein the first strain uses the Wood-Ljungdahl (WL) pathway to fix a carbon oxide.

34. The method according to claim 33, wherein the first strain is at least one of Clostridium coskatii, Clostridium ljungdahlii, Clostridium autoethanogenum, Clostridium ragsdalei, Terrisporobacter glycolicus, and Clostridium scatologenes.

35. The method according to claim 31, wherein the second strain is at least one of Clostridium cadaveris, Clostridium sporogenes, Clostridium sticklandii, Clostridium propionicum, Clostridium botulinum, and Clostridium pasteurianum.

36. The method according to claim 31, wherein the third strain is a Clostridium sp. strain.

37. The method according to claim 36, wherein the third strain is at least one of Clostridium tyrobutyricum, Clostridium butyricum, Clostridium beijerinckii, Clostridium acetobutylicum, Clostridium argentinense, Clostridium aurantibutyricum, Clostridium botulinum, Clostridium carboxidivorans, Clostridium cellulovorans, Clostridium cf. saccharolyticum, Clostridium difficile, Clostridium kluyveri, Clostridium novyi, Clostridium paraputrificum, Clostridium pascui, Clostridium peptidivorans, Clostridium perfringens, Clostridium scatologenes, Clostridium schirmacherense, Clostridium sticklandii, Clostridium subterminale SB4, Clostridium symbiosum, Clostridium tetani, Clostridium tepidiprofundi Clostridium tertium, Clostridium tetanomorphum, and Clostridium thermopalmarium.

38. The method according to claim 31, wherein the microorganism co-culture system further comprises a co-substrate being lactic acid.

39. The method according to claim 38, wherein the co-substrate is a lactic acid and the saccharide and the lactic acid are used at a weight ratio of saccharide: lactic acid=1:1 to 1:10.

40. The method according to claim 31, wherein the chemical conversion reaction is at least one of catalytic hydrogenation and esterification-hydrogenolysis.
